(19) United States
(12) Patent Application Publication
(10) Pub. No.: US 2023/0112882 A1
(43) Pub. Date: Apr. 13, 2023

(54) DEVICES, SYSTEMS, AND METHODS OF APPLYING NANO-DIMENSIONED ICE PARTICLES TO ANIMAL TISSUES

(71) Applicant: Glacia, Inc., Snohomish, WA (US)

(72) Inventor: Craig Clinton Rominger, Snohomish, WA (US)

(73) Assignee: Glacia, Inc., Snohomish, WA (US)

(21) Appl. No.: 18/046,472

(22) Filed: Oct. 13, 2022

Related U.S. Application Data
(60) Provisional application No. 63/255,350, filed on Oct. 13, 2021.

(51) Int. Cl. A61F 7/00

(52) U.S. Cl. CPC A61F 7/0085; A61F 2007/0024; A61F 2007/0056; A61F 2007/0096

(57) ABSTRACT

A therapeutic device includes a substrate, a seal to couple the substrate to a surface, and at least one port defined in the substrate through which a therapeutic fluid is introduced between the surface and the substrate such that the therapeutic fluid directly contacts the surface.

### RELATED APPLICATIONS

This application claims priority to and incorporates U.S. Provisional Pat. Application 63/255,350, filed Oct. 13, 2021, entitled “Temperature-Dependent Applications of Nano-Dimensioned Ice Particles and Methods Thereof,” in its entirety by reference.

### TECHNICAL FIELD

The present disclosure relates generally to therapeutic devices and methods for animals. Specifically, the present disclosure relates to systems and methods for applying a nano-dimensioned ice crystal slurry to a target animal tissue.

### BACKGROUND

Animals including all vertebrates such as humans, horses, and other animals may overuse or injure tissues including muscle tissues. Icing an overused or injured tissue has been broadly used since 1978 when Dr. Gabe Mirkin coined the term RICE (Rest, Ice, Compression, Elevation). Icing has been used to reduce inflammation of tissues to accelerate healing and speed of recovery. In the past, the ice has been applied in a towel or put in a treatment device with a barrier to protect the skin from frostbite and further damaging the skin. This ineffective process is described in, for example, U.S. Pat. No. 5,887,437 and U.S. Pat. Application Publication No. 2018/0000636 where the therapy device is held to the skin with a barrier between the skin or other tissue and the ice.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates a wrap for use with a therapeutic fluid, according to an example of the principles described herein.

FIG. 2 illustrates a wrap for use with a therapeutic fluid, according to an example of the principles described herein.

FIG. 3 illustrates a wrap for use with a therapeutic fluid and for an elbow of a human, according to an example of the principles described herein.

FIG. 4 illustrates a wrap for use with a therapeutic fluid and for a cannon and fetlock of a horse, according to an example of the principles described herein.

FIG. 5 illustrates a wrap for use with a therapeutic fluid and for a cannon and fetlock of a horse, according to an example of the principles described herein.

FIG. 6 illustrates a wrap system for use with a therapeutic fluid and for a forearm, cannon, and fetlock of a horse, according to an example of the principles described herein.

FIG. 7 illustrates a pair of wraps for use with a therapeutic fluid and for a forearm, cannon, and fetlock of a horse, according to an example of the principles described herein.

FIG. 8 illustrates the pair of wraps of FIG. 7, according to an example of the principles described herein.

FIG. 9 illustrates a pair of wraps for use with a therapeutic fluid and for a forearm, cannon, and fetlock of a horse, according to an example of the principles described herein.

FIG. 10 illustrates a pair of wraps for use with a therapeutic fluid and for a forearm, cannon, and fetlock of a horse, according to an example of the principles described herein.

FIG. 11 illustrates a pair of wraps for use with a therapeutic fluid and for a fetlock, a pastern, a heel, and a hoof of a horse, according to an example of the principles described herein.

FIG. 12 illustrates a protective cover for use with a therapeutic fluid and for a foot of a human, according to an example of the principles described herein.

FIG. 13 illustrates a wrap for use with a therapeutic fluid and for the knee area of a human, according to an example of the principles described herein.

FIG. 14 illustrates a wrap for use with a therapeutic fluid and for the knee area of a human, according to an example of the principles described herein.

FIG. 15 illustrates a pair of wraps for use with a therapeutic fluid and for the knee area of a human, according to an example of the principles described herein.

FIG. 16 illustrates a patch for use with a therapeutic fluid, according to an example of the principles described herein.

FIG. 17 illustrates a patch for use with a therapeutic fluid and for the application to an area of a human body, according to an example of the principles described herein.

FIG. 18 illustrates a front perspective view of a patch, according to an example of the principles described herein.

FIG. 19 illustrates a top perspective view of the patch of FIG. 18, according to an example of the principles described herein.

FIG. 20 illustrates a rear perspective view of the patch of FIG. 18, according to an example of the principles described herein.

FIG. 21 illustrates a close-up, rear perspective view of the patch of FIG. 18, according to an example of the principles described herein.

FIG. 22 illustrates a side view of the patch of FIG. 18, according to an example of the principles described herein.

FIG. 23 illustrates an exploded view of a patch, according to an example of the principles described herein.

FIG. 24 illustrates a front perspective view of a patch, according to an example of the principles described herein.

FIG. 25 illustrates a pair of patches applied to the back of a human, according to an example of the principles described herein.

FIG. 26 illustrates the patch of FIG. 25 as applied to a shoulder of a horse, according to an example of the principles described herein.

FIG. 27 illustrates a side perspective view of a patch, according to an example of the principles described herein.

FIG. 28 illustrates a top perspective view of a patch, according to an example of the principles described herein.

FIG. 29 illustrates a front view of a patch, according to an example of the principles described herein.

FIG. 30 illustrates a patch for use with a therapeutic fluid, according to an example of the principles described herein.

FIG. 31 illustrates a front view of a patch, according to an example of the principles described herein.

FIG. 32 illustrates a front perspective view of the patch of FIG. 31 including a retention clip, according to an example of the principles described herein.

FIG. 33 illustrates a front perspective view of a pair of patches of FIG. 31 coupled together, according to an example of the principles described herein.

FIG. 34 illustrates a close-up, front perspective view of the patch of FIG. 31 with a retention clip removed, according to an example of the principles described herein.

FIG. 35 illustrates a front perspective view of a patch, according to an example of the principles described herein.

FIG. 36 illustrates a side, cutaway view of the patch of FIG. 35, according to an example of the principles described herein.

FIG. 37 illustrates front view of the patch of FIG. 35, according to an example of the principles described herein.

FIG. 38 illustrates a front perspective view of a patch, according to an example of the principles described herein.

FIG. 39 illustrates a side, cutaway view of the patch of FIG. 38, according to an example of the principles described herein.

FIG. 40 illustrates front view of the patch of FIG. 38, according to an example of the principles described herein.

### DESCRIPTION OF EXAMPLE EMBODIMENTS

### Overview

Notably, the current marketplace for cooling acute injury and reducing inflammation does not properly address deep tissue penetration which is believed to be a superior way to reduce internal inflammation of, for example, muscle tissues. Research and testing has demonstrated that in the known industry, most ice that is used is too cold, hard, dendritic, and lacks certain heat transfer properties to produce an optimal therapeutic response. Skin prevents most ice modalities from being effective; and using the skin as a transfer agent is one of the best ways to remove heat from the body and effectively reduce inflammation.

Human fat cells begin to die off as temperatures drop below 40° F. Cryolipolysis, commonly referred to as “CoolSculpting” by patients, uses cold temperature to break down fat cells. The fat cells are particularly susceptible to the effects of cold, unlike other types of cells. While the fat cells freeze, the skin, muscle, and other tissues and structures are spared from injury. Thus, additionally, a market of fat cell reduction via cooling of tissues exists. However, this process appears to be a violent and brutal process for patients. Though U.S. Food and Drug Administration (US FDA) approved of the use of cryolipolysis procedures, and these processes are valued in the market near +/- $15 billion, many in the cryolipolysis industry are currently being sued for malpractice due to damage to patients and pain and suffering from such treatments.

In the examples described herein, a therapeutic fluid may be used to treat damaged animal tissues such as, for example, the epidermis, the dermis, the subcutis, the hypodermis, muscles, ligaments, bones, internal organs, and other animal tissues. For example, a human individual may have a deep tissue injury such as bruising or a strain. However, currently there does not exist a system or method of properly cooling this and other types of injury in order to reduce inflammation in the tissues. Particularly, deep tissue penetration of a cooling effect, which is believed to be a superior method of truly reducing internal inflammation, is not provided in the current marketplace.

Testing has demonstrated that the use of conventional ice is ineffective and potentially dangerous. For example, most ice used in therapeutic applications is too cold. Further, ice is hard, dendritic, and lacks certain heat transfer properties to make it optimal in effecting relatively deeper tissues since the skin of the animal prevents most ice modalities from effectively transferring heat. However, using the skin as a transfer agent is one of the best ways to remove heat from the body, and effectively reduce inflammation in the skin and in tissues internal to the skin.

Further, ice has been used for certain bariatric treatments. Human fat cells, for example, begin to die off as temperatures drop below 40° F. Thus, additionally, a market of fat cell reduction by cooling exists. However, in the process currently used referred to as cryolipolysis (e.g., “cool sculpting”), cold temperatures may be used to kill fat cells. However, this process may be a violent and brutal process for patients. Though FDA approved and valued in the market at approximately $15 billion, many in the cryolipolysis industry are currently being sued for malpractice due to injuries sustained during these processes.

Thus, examples described herein provide for a therapeutic device including a substrate, a seal to couple the substrate to a surface to be treated, and at least one port defined in the substrate through which a therapeutic fluid may be introduced between the surface to be treated and the substrate such that the therapeutic fluid directly contacts the surface. The therapeutic fluid may include a nano-dimensioned ice crystal slurry that is not harmful in sustained use and is design to be used directly on the skin of the animal such that the cooling effect of the nano-dimensioned ice crystal slurry is able to penetrate to deep tissues within the body of the animal.

This disclosure is directed to the multiple ways in which application of nano-dimensioned ice particles that are applied within a particular temperature range are able to provide superior cooling effects for various purposes. When compared to using a standard form of ice, the effects found using the nano-dimensioned ice slurry described herein is superior to the effects achieved by the standard ice for the same process. The ice crystals may have a width ranging from approximately 100 nm to 500 nm, where the term “approximately” may be understood to include plus or minus 15%. In an embodiment, for example, 250 non-dimensioned ice crystals may be contained in a space that is a sized roughly equivalent to an average width of a tip of a human hair.

The temperature of the ice crystals may assist in achieving quality, effective, and improved results for many processes. The temperature of standard ice, (e.g., cubed, block, chunk, pebble, crushed, etc.) is too cold for any procedure. In on example, the temperatures for ice crystal application for all human and animal applications is not 0° F. Rather, it is warmer than 0° F. In one example, the range for any human of animal application, whether internal, external, or ingested, may range from 24.5° F. to 35° F., and in some instances may fluctuate as much as 10% on the low or high end. Additionally, in an embodiment, the temperature range indicated above may range from 26° F. to 31° F.

Further, the structure of the ice crystals may be important, as those skilled in the art may appreciate that making ice within the relatively warm temperature ranges mentioned above is extremely difficult. To make ice at the above-mentioned temperatures, the structure of the ice crystal is changed from the standard structures, as reliance on historical ice physics limits development and accuracy. The ice structure for the ice crystals described herein may produce non-random, 100% consistent ice product. The ice structure for ice crystals described herein may be the same size, structure, and temperature, depending on the application.

The potential uses of the nano-sized ice particles may be extremely vast. For example, the nano-sized ice particles may be used to treat acute injuries where the temperature of the nano-sized ice particles may range from about 28° F. to about 29.5° F. Another application of the nano-sized ice particles may include a patient with chronic arthritis, aged 55 \+ years were the temperature of the nano-sized ice particles may range from approximately 30.5° F. to approximately 31° F.

The nano-sized ice particles may be may also be used in connection with fat cell reduction. Because the nano-sized ice particles may be non-abrasive, and do not damage the skin, and will not cause long-term damage when applied appropriately, the nano-sized ice particles may be used in certain fat cell reduction processes. The nano-sized ice particles may be able to maintain the temperature in a consistent, effective range for many hours, and up to several days in some applications. When applied directly to skin, with no force, suction or other machinery, the nano-sized ice particles are able to reduce the temperature of subcutaneous fat cells below 40° F., in less than 20 minutes. Multiple applications have shown to kill fat cells over the subsequent 1-3 months. These processes using the nano-sized ice particles may be similar to cryolipolysis, but do not present any lingering side effects and provide for a short recovery period (e.g., approximately 3-5 days) as compared to weeks or months from current methods. In one example, the nano-sized ice particles may be consistently applied over the intended area at a static temperature of 28° F., for a period of 20 minutes, in one or separate treatments 30 days apart.

Other uses of the nano-sized ice particles may include the treatment of animals (e.g., horses, dogs, cattle, etc.) including inflammation reduction to minimize risk of further uncontrollable injury. A sample of other possible example uses and temperature ranges may include cooling organs in situ (about 26° F. to about 27.5° F.), ex situ cooling and preparation (about 28.5° F. to about 29.5° F.), organ transport cooling and maintenance (about 27.5° F. to about 30° F.), and organ shelf-life extension (about 28° F. to about 29.5° F.), among many other uses. The nano-sized ice particles may be placed in direct contact with tissues of an animal. This direct interaction between the nano-sized ice particles and the tissues may provide greater efficacy in reducing and/or eliminating swelling, reducing and/or eliminating pain, and increasing speed of and chances for recovery, among a myriad of other advantages.

Examples described herein also provide a therapeutic device may include a substrate, a seal to couple the substrate to a surface to be treated, and at least one port defined in the substrate through which a therapeutic fluid may be introduced between the surface to be treated and the substrate such that the therapeutic fluid directly contacts the surface. The surface to be treated may include one or more layers of outer tissue of a vertebrate animal. The therapeutic fluid may include a nano-dimensioned ice crystal slurry. The seal may include a suction device that creates negative fluid pressure between the substrate and the surface to create a partial vacuum between the substrate and the surface to be treated. The therapeutic device may further include a pressure regulator to selectively release pressure within the suction device. The substrate may be made of an elastic material to conform to a contour shape of the surface to be treated.

The at least one port may include a first port through which the therapeutic fluid may be introduced between the surface to be treated and the substrate, and a second port through which the therapeutic fluid may be drained from between the surface to be treated and the substrate.

The therapeutic device may further include a temperature readout device disposed ont eh substrate. The therapeutic device may further include a notification device to produce one or more notifications associated with use of the therapeutic device. The therapeutic device may further include at least one sensor disposed on the substrate and a communication device to communicate data obtained from the sensor. The therapeutic device may further include at least one coupling device to couple the therapeutic device to another therapeutic device.

Examples described herein also provide a therapeutic method. The therapeutic method may include applying a therapeutic device to a surface to be treated. The therapeutic device may include a substrate, a seal to couple the substrate to the surface to be treated, and at least one port defined in the substrate through which a therapeutic fluid may be introduced between the surface to be treated and the substrate such that the therapeutic fluid directly contacts the surface. The method may further include introducing the therapeutic fluid between the surface to be treated and the substrate via the at least one port.

The therapeutic method may further include coupling the substrate to the surface to be treated via the seal, the seal comprising, a suction device, an adhesive, hook and loop (e.g., Velcro®), tape, and combinations thereof. The therapeutic method may further include creating negative fluid pressure between the substrate and the surface to create a partial vacuum between the substrate and the surface to be treated.

The therapeutic method may further include, with a pressure regulator, selectively releasing pressure within the suction device. The therapeutic method may further include forming the therapeutic fluid, the therapeutic fluid comprising a nano-dimensioned ice crystal slurry.

The at least one port may include a first port through which the therapeutic fluid may be introduced between the surface to be treated and the substrate, and a second port through which the therapeutic fluid may be drained from between the surface to be treated and the substrate. The therapeutic method may further include removing the therapeutic fluid via the second port.

The therapeutic method may further include detecting, via at least one sensor coupled to the therapeutic device, at least one environmental characteristic associated with the therapeutic method, and transmitting, via a communication device associated with the therapeutic device, data defining the environmental characteristic.

The at least one sensor may include a temperature sensor. The communication device may transmit data defining a temperature of the therapeutic fluid. The therapeutic method may further include coupling the therapeutic device to another therapeutic device via at least one coupling device.

Additionally, the techniques described in this disclosure may be performed as a method and/or by a system having non-transitory computer-readable media storing computer-executable instructions that, when executed by one or more processors, performs the techniques described above.

As used in the present specification and in the appended claims, the term “animal” is meant to be understood broadly as any animal taxa within the subphylum Vertebrata (chordates with backbones), including all mammals, birds, reptiles, amphibians, and fish. Several examples herein describe the systems and methods used on connection with the therapeutic treatment of human and horse tissues. However, any animal of the subphylum Vertebrata may benefit from the systems and methods described herein.

As used in the present specification and in the appended claims, the term “therapeutic fluid” is meant to be understood broadly as any solid, liquid, slurry, or combinations thereof that may be used to provide therapeutic benefits to an animal. The therapeutic fluid may include a nano-dimensioned ice crystal slurry that is able to penetrate the skin of the animal. The therapeutic fluid produced at this nanoscale also allows for dilation, reduction of swelling, and opening of pores of the user’s skin to allow possible therapeutic agents in the nano-ice, frozen cryogenic fluid or slurry to pass into the skin topically. Similar effects may be experienced in connection with different types of tissues and organs. These therapeutic agents in the nano-ice, frozen cryogenic fluid or slurry may include, for example, methylsulfonylmethane (MSM), glucosamine, aloe including pure aloe, Epsom salts, trehalose, autologous cultured chondrocytes, cytokines for wound healing (e.g., derma gel, silvasorb, chlorhexidine 2%/4%, steroid creams), botulinum toxin type A, onabotulalinumtoxina (e.g., Botox), baclofen, tizanidine, cyclobenzaprine, iodine preparations (e.g., tincture of iodine, potassium iodide, iodophors), copper preparations (e.g., copper sulfate, copper naphthenate, cuprimyxin), sulfur preparations (e.g., monosulfiram, benzoyl disulfide), phenols (e.g., phenol, thymol), fatty acids and salts (e.g., propionates, undecylenates), organic acids (e.g., benzoic acid, salicylic acids), dyes (e.g., crystal [gentian] violet, carbolfuchsin), hydroxyquinolines (e.g., iodochlorhydroxyquin), nitrofurans (e.g., nitrofuroxine, nitrofurfurylmethyl ether), imidazoles (e.g., miconazole, tioconazole, clotrimazole, econazole, thiabendazole), polyene antibiotics (e.g., amphotericin B, nystatin, pimaricin, candicidin, hachimycin), allylamines (e.g., naftifine, terbinafine), thiocarbamates (e.g., tolnaftate), and miscellaneous agents (e.g., acrisorcin, haloprogin, ciclopirox, olamine, dichlorophen, hexetidine, chlorphenesin, triacetin, polynoxylin, amorolfine, Triclosan, Microban, Iodine, O-phenylphenol, Hydronium, Dakin’s Solution, hydrogen peroxide, honey, vinegar, essential oils, Erythromycin (e.g., antibiotics), mesenchymal stem cells (e.g., MSCs), platelet-rich plasma (PRP), autologous conditioned serum (ACS) and autologous protein solution (APS), chlorhexidine, dermatophilus congolensis, and combinations thereof, among other chemical compositions.

In one example, the cryogenic fluid composition may be formulated within an auger system to allow for one or more formations including dendrites, plates, solid prisms, hollow prisms, solid columns, hollow columns, and needles, among other formations. In one example, the formations may be generated along an interior wall of a core surrounding an auger of the auger system at between approximately 2° to -5° C. range (approximately 35° F. to 23° F.). At this range of temperatures, the formations may be scraped or knocked off the interior wall. In one example, the cryogenic fluid composition may be formulated as a supersaturation in grams per meter cubed (g/m3) at approximately 0 to 0.3 g/m3. The formation of the cryogenic fluid or slurry at these temperatures and supersaturation levels allows for the formulations described herein to form rather than, for example, relatively larger formulations. As the formations are scraped or knocked off the interior wall, the formations may be subjected to shear forces that create even smaller formations such as the nano-ice formations described herein. Thus, in the first instance of creation, the formations may be relatively smaller, and the formations further decrease in size as they are scraped or knocked off the interior wall.

As used in the present specification and in the appended claims, the term “adhesive” is meant to be understood broadly as any substance that couples a first element to a second element. In one example, the adhesive may permanently, semi-permanently, or temporarily couple the first element to the second element. In examples described herein, the adhesive may serve to create a fluid tight seal between a wrap or patch and the outer portions of the tissues of an animal. In one example, the adhesive may include any composition capable of coupling the wrap or patch to a surface of the tissues such as, for example, skin, hair, organs, etc. to be treated. In one example, the adhesive may include silicone, polyurethane (PU) gel adhesives, acrylics, water-based adhesives, water-soluble adhesives, pressure-sensitive adhesives, pressure-activated adhesives, heat-reactive adhesives, contact adhesives, medical-grade adhesives, topical adhesives, other types of adhesives, and combinations thereof. In one example, the adhesive may include a Silbione™ silicone adhesive manufactured and distributed by Elkem.

### EXAMPLE EMBODIMENTS

### Examples of Wrap Devices

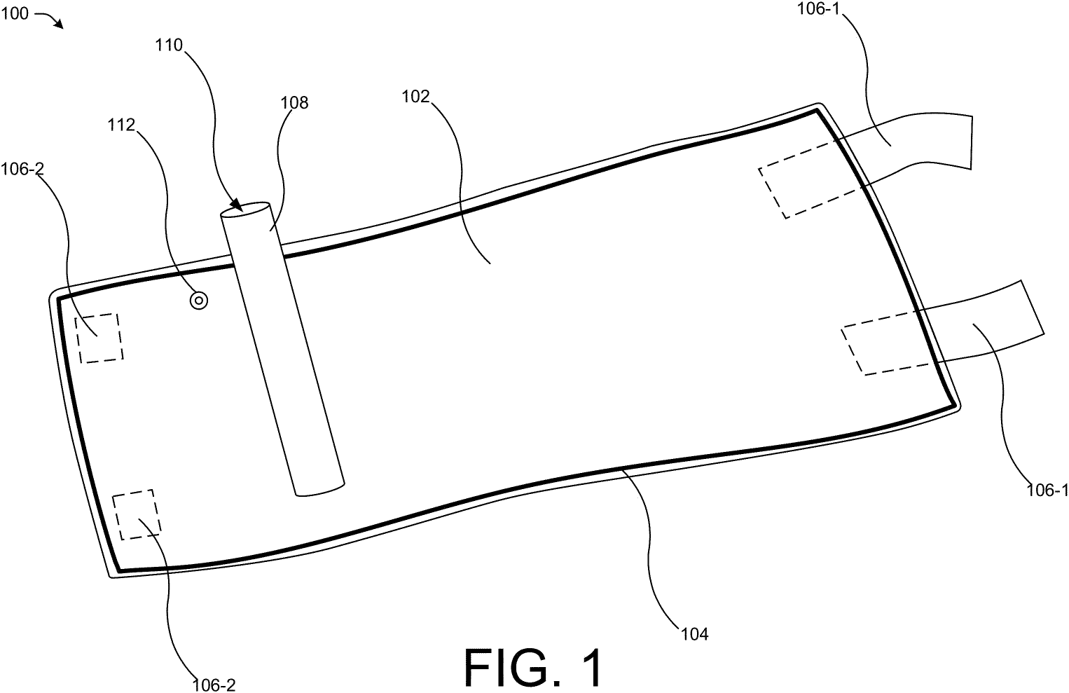

In examples described herein in connection with FIGS. 1 through 15, several wraps are described. The wraps are used to ensure direct contact of a therapeutic fluid with the skin of an animal. In the past, ice and other frozen substances have been used indirectly to cool injured tissues. However, this indirect therapy is ineffective and may result in the injured tissues not receiving an appropriate level of therapy.

Turning now to the figures, FIG. 1 illustrates a wrap 100 for use with a therapeutic fluid, according to an example of the principles described herein. The wrap 100 may be wrapped around any portion of an animal’s core or extremities such as an arm or leg. The wrap 100 may include a substrate 102 that may be used to retain the therapeutic fluid therein. The substrate 102 may be made of any material that may be wrapped around tissues, and therefore may be made of a fabric, a plastic, a rubber, or other bendable material. Further, the substrate 102 may be made of a material capable of retaining fluids such as the therapeutic fluid is retained between the substrate 102 and the outer portions of the tissues of the animal. In this manner and in the examples described herein, the therapeutic fluid is kept in direct contact with the animal tissues. In one example, the substrate 102 may be made of a waterproof or water resistant polyester material.

An adhesive 104 may be formed onto or applied to one or more edges of the substrate 102 to allow for the substrate 102 to adhere to treated tissues of the animal (e.g., skin). The adhesive serves to create a fluid tight seal between the substrate 102 and the outer portions of the tissues of the animal. In this manner, the therapeutic fluid may be contained against the tissues of the animal.

A fastener such as hook-and-loop fasteners (e.g., Velcro®) with one or more straps of hooks 106-1 coupled to a first end of the substrate 102, and a corresponding number of straps of loops 106-2 coupled to a second end of the substrate 102. In this manner, coupling of the straps of hooks 106-1 and the straps of loops 106-2 causes the first end and the second end to be coupled to one another and retain the substrate against the tissues of the animal.

A port 108 including an orifice 110 may be coupled to the substrate 102 or retained within the substrate 102. The port 108 may act as a conduit through which the therapeutic fluid may be piped into the space between the substrate 102 and the outer portions of the tissues of the animal.

The wrap 100 of FIG. 1 may further include a pressure release valve 112. The pressure release valve 112 may be used to allow air to escape the space between the substrate 102 and the outer portions of the tissues of the animal as the therapeutic fluid is introduced into the space. Once the therapy is complete, the adhesive 104 may be released from the tissues of the animal and the therapeutic fluid may fall from the space between the substrate 102 and the outer portions of the tissues of the animal. In one example, the wrap 100 may further include a dispensing port to dispense the therapeutic fluid from the space between the substrate 102 and the outer portions of the tissues of the animal so that the therapeutic fluid does not contaminate other items.

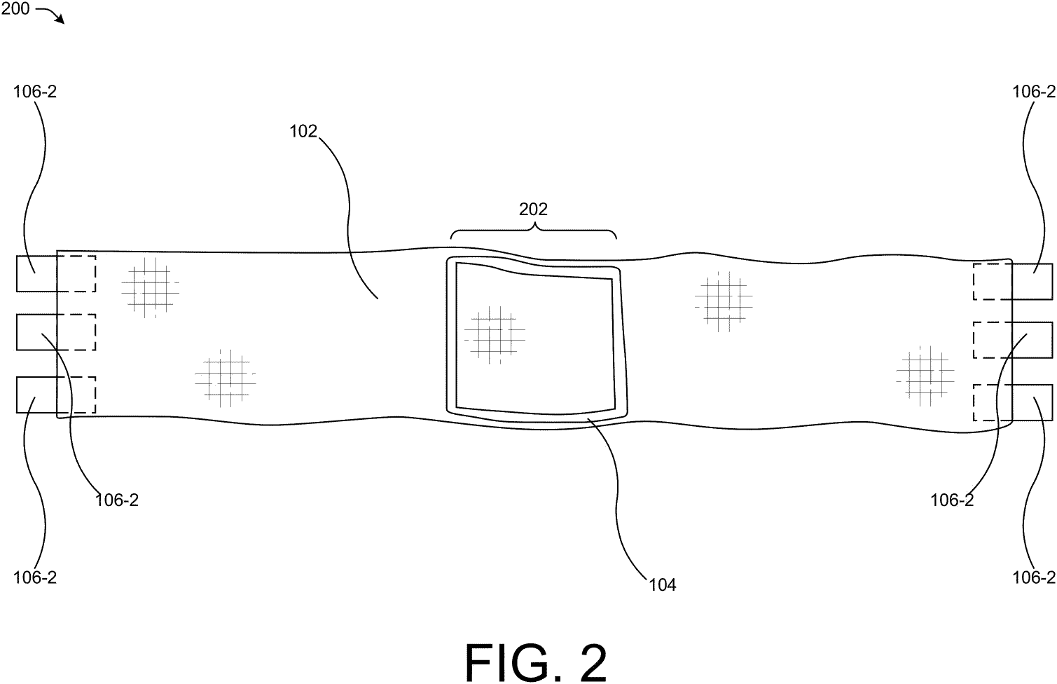

FIG. 2 illustrates a wrap 200 for use with a therapeutic fluid, according to an example of the principles described herein. The example of FIG. 2 may include similar elements as included in the example of FIG. 1 including the substrate 102, the adhesive 104, the straps of hooks 106-1, and the straps of loops 106-2. In the example of FIG. 2, a therapeutic area 202 may be relatively smaller than the substrate 102. The substrate 102 may be wrapped around a relatively larger area such as a torso of an animal while allowing for the therapeutic area 202 to be placed in a specific area of the animal. The therapeutic fluid may be introduced into the therapeutic area 202 using, for example, port 108 as depicted in the example of FIG. 1.

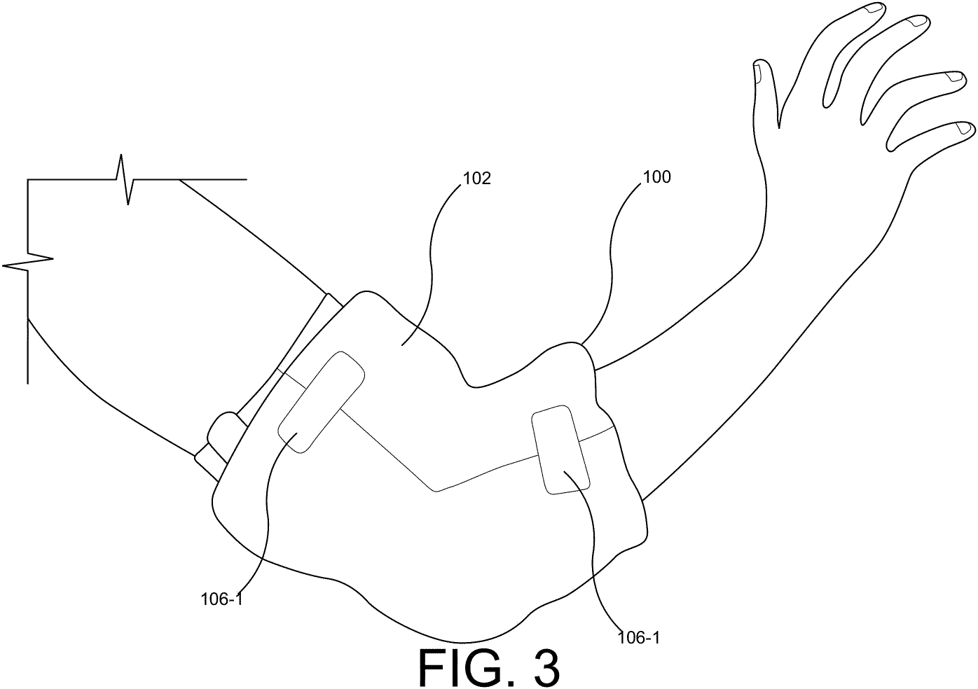

FIG. 3 illustrates a wrap 100 for use with a therapeutic fluid and for an elbow of a human, according to an example of the principles described herein. As depicted in the example of FIG. 3, the wrap 100 such as the example of FIG. 1 may be wrapped around any portion of a part of the animal such as, for example, an elbow and arm of a human individual. The wrap 100 may be left on the arm of the user in the manner depicted in FIG. 3 for a period of time to allow for the therapeutic fluid to sufficiently cool the tissues to be treated.

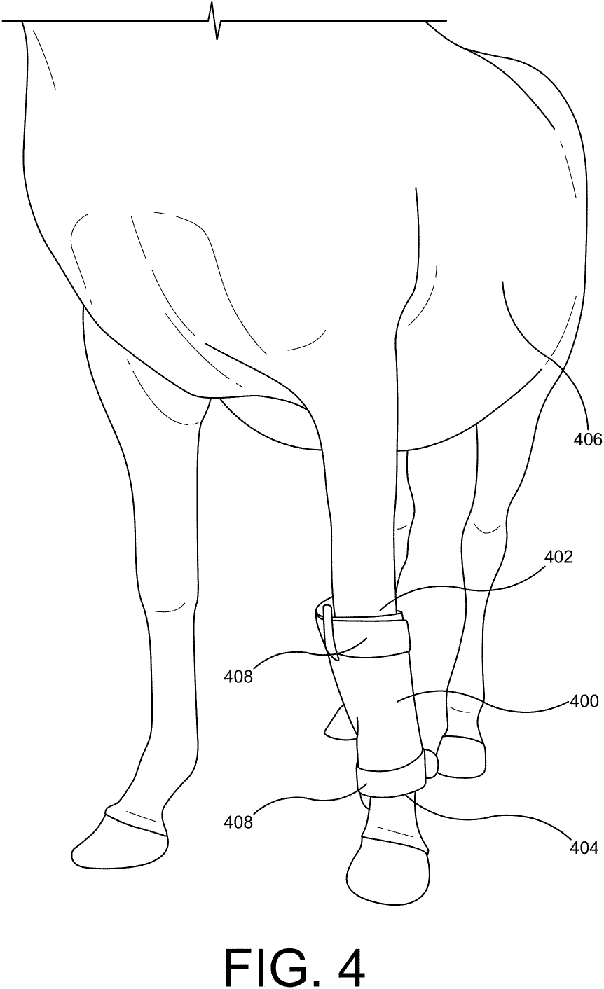

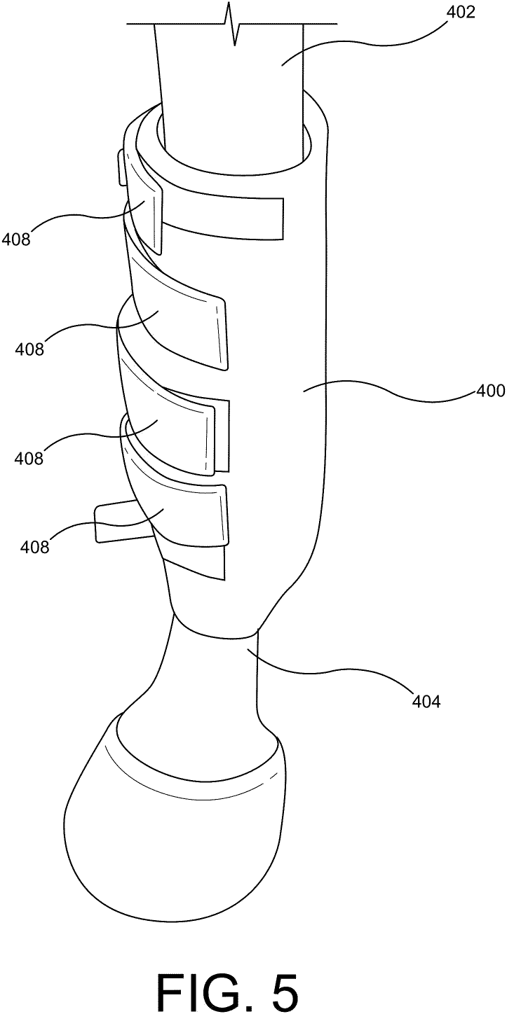

FIG. 4 illustrates a wrap for use with a therapeutic fluid and for a cannon 402 and fetlock 404 of a horse 406, according to an example of the principles described herein. FIG. 5 illustrates the wrap 400 for use with a therapeutic fluid and for the cannon 402 and the fetlock 404 of the horse 406, according to an example of the principles described herein. Animals other than humans can also benefit from the therapeutic systems and methods described herein. In many instances, a horse, for example, may become injured or may overuse a muscle or ligament that may cause the horse to, at least temporarily, become lame. Horse trainers, stable owners, and veterinarians may improve equine lower-limb cooling therapy to prevent and treat injuries. The therapeutic fluid may be used to cool equine lover limbs and stifle joint and hoof laminae at lower temperatures for longer periods of time.

In order to couple the wrap 400 to the cannon 402 and fetlock 404 of the horse 406, a couple of straps 408 may be used to cause the adhesive 104 (FIGS. 1 and 2) to properly adhere to the tissues of the animal including hair, fur, skin, etc. In one example, the wrap 400 of FIGS. 4 and 5 may be made of a heavy duty nylon fabric and may include any type of fastener for use as the straps 408 including, for example, hook-and-loop fasteners, dual lock fasteners, adhesives zippers, snap coupling devices, other types of fasteners and combinations thereof. In one example, one or more compression rods may be incorporated into the wrap 400 and run vertically up one or more sides of the wrap 400 to ensure that space is maintained between the wrap 400 and the surface of the body of the horse 406.

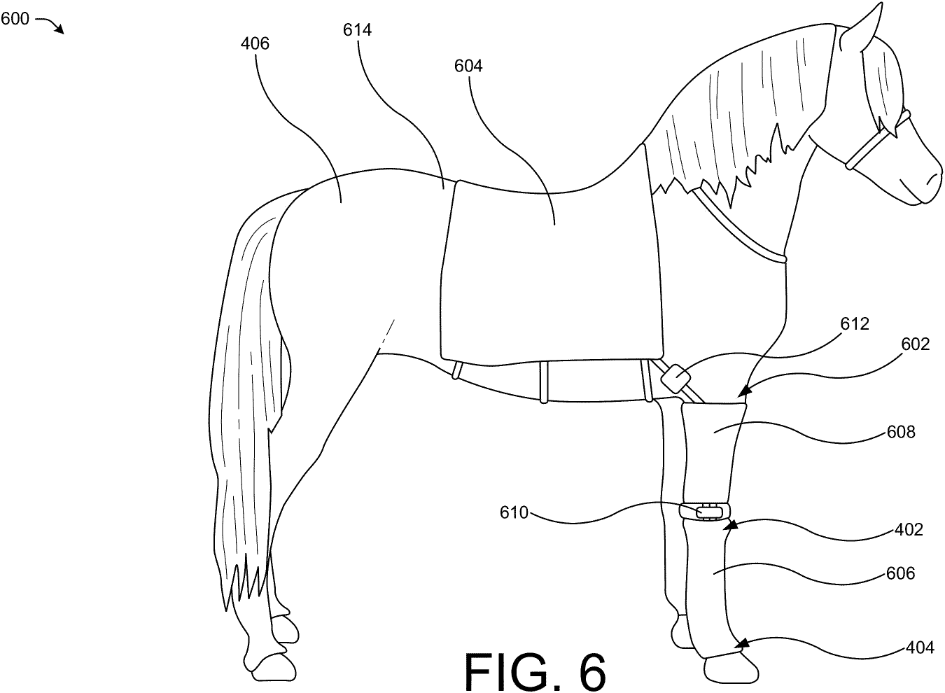

FIG. 6 illustrates a wrap system 600 for use with a therapeutic fluid and for a forearm 602, cannon 402, fetlock 404 and torso 614 of a horse 406, according to an example of the principles described herein. The wrap system 600 may be used to cover relatively more of the horse 406 to provide several points of therapeutic treatment. For example, the wrap system 600 may include a first wrap 606 to treat the cannon 402 and/or fetlock 404 of the horse 406, a second wrap 608 to treat the forearm 602 and/or cannon 402 of the horse 406, and a third wrap 604 to treat the torso 614. The third wrap 604 may be used to cool both the skin and muscles located on the torso 614 and may also be used to cool internal organs of the horse 406 including, for example, the heart of the horse 406. The elements of the wraps as described in FIGS. 1 through 5 may be incorporated into the first wrap 606, the second wrap 608, and the third wrap 604.

In the example of FIG. 6, the first wrap 606 and the second wrap 608 may be coupled together via a first coupling device 610 such as a buckle, strap, etc. Thus, in addition to any straps 408 that may be used to couple the first wrap 606 and the second wrap 608 to the horse 406, the first coupling device 610 may be used to ensure that the first wrap 606 and the second wrap 608 do not fall down the leg of the horse 406. Further, the wrap system 600 may include a second coupling device 612 to couple the second wrap 608 to the third wrap 604. Thus, like the first coupling device 610, the second coupling device 612 serves to ensure that the first wrap 606 and/or the second wrap 608 do not fall down the leg of the horse 406 as they are directly or indirectly coupled to the third wrap 604 that is securely anchored to the torso 614 of the horse 406.

For each of the first wrap 606, the second wrap 608, and the third wrap 604 may be coupled to the hair and/or skin of the horse 406 via the adhesive 104. The space between each of the first wrap 606, the second wrap 608, and the third wrap 604 and the hair and/or skin of the horse 406 may be filled with the therapeutic fluid via a port 108 as described herein. In this manner, one or more areas of the body of an animal such as a human or a horse may be simultaneously treated.

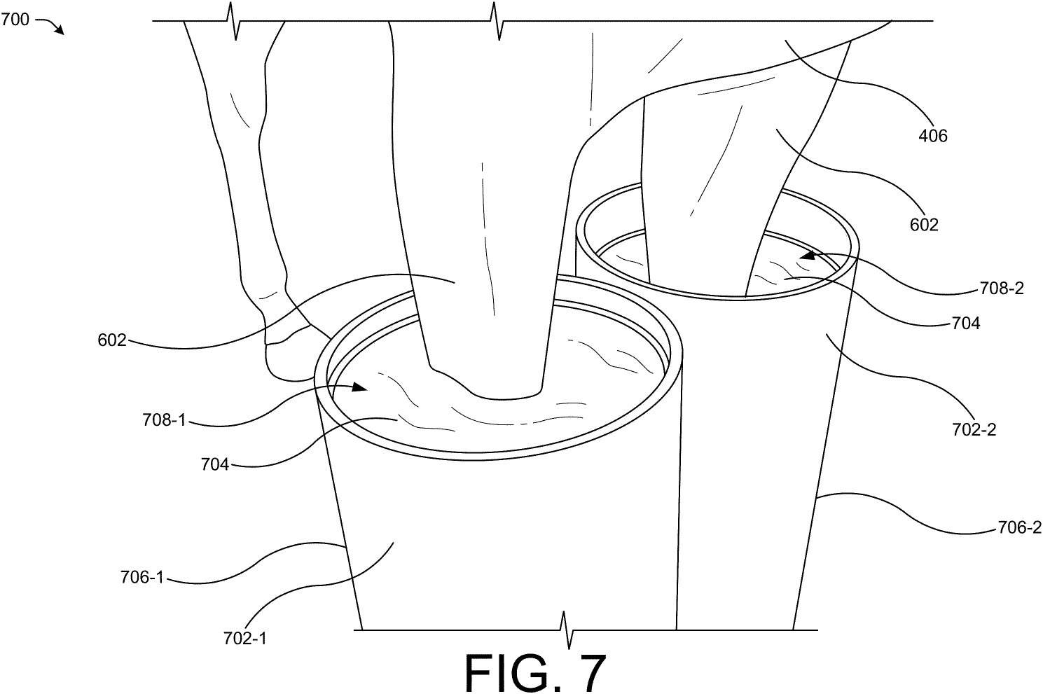

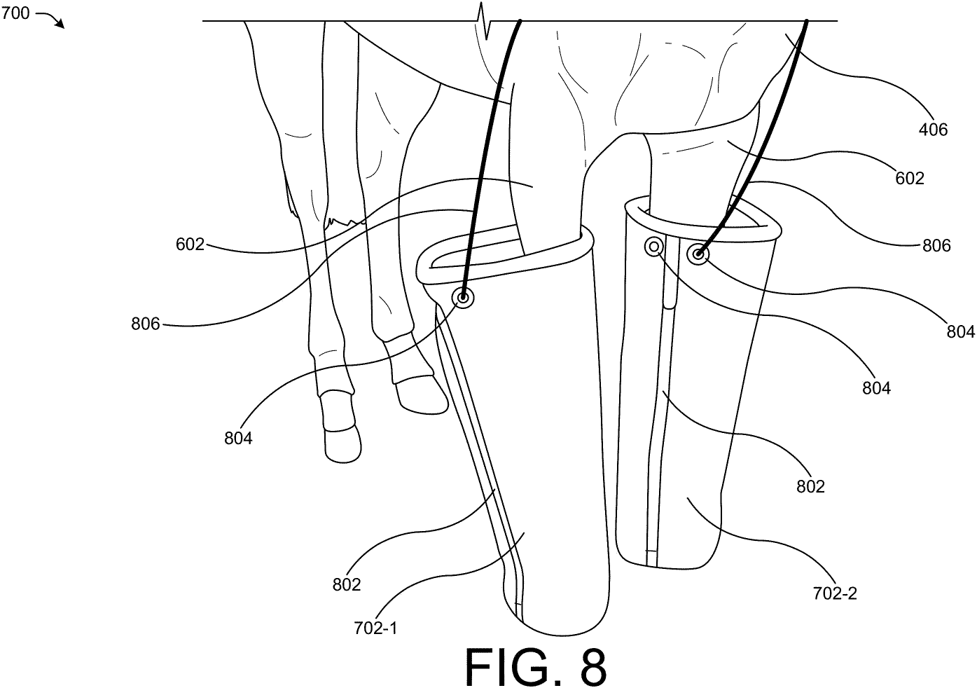

FIG. 7 illustrates a pair of wraps 700 for use with a therapeutic fluid and for a forearm 602, cannon (not shown), and fetlock (not shown) of a horse 406, according to an example of the principles described herein. FIG. 8 illustrates the pair of wraps 700 of FIG. 7, according to an example of the principles described herein. The pair of wraps 700 may include a first wrap 702-1 for a front, right leg of the horse 406 and a second wrap 702-2 for a front, left leg of the horse 406 and may have the form factor of a boot or well into which the legs of the horse 406 are inserted. However, any number of wraps may be used in connection with the treatment of an animal including back left and back right wraps for the horse 406 as well. The wraps 702-1, 702-2 may include a well-type form factor including an outer housing 706-1, 706-2 with an opening 708-1, 708-2 to allow for the insertion of a therapeutic fluid 704 such as a nano-dimensioned ice crystal slurry. A well-type form factor may include any form factor that allows for the filling of the space between the wraps 702-1, 702-2 and the tissues of the animal wherein gravity is used to hold the therapeutic fluid 704 within the wrap(s). The wraps 702-1, 702-2 may further include one or more fasteners 802 along a length of the wraps 702-1, 702-2 such as, for example, one or more zippers coupled to two edges of a horizontal opening. In one example, the zippers or other fasteners may be waterproof to ensure that the therapeutic fluid 704 does not leak outside the wraps 702-1, 702-2. Further, the fasteners 802 may be used to accommodate for the insertion of a body part into the wraps 702-1, 702-2 such as a forearm 602, cannon 402, fetlock 404, and/or hoof of a horse 406 via the opening. In this manner, the wraps 702-1, 702-2 may cause the therapeutic fluid to contact the skin of the horse 406 to ensure a direct contact with the therapeutic fluid 704 as the nano-dimensioned ice crystal slurry penetrates the skin and other tissues to deliver a superior therapeutic effect.

The wraps 702-1, 702-2 of FIGS. 7 and 8 may be made of a relatively rigid material such as, for example, a rubber, a polyvinyl chloride (PVC), a halogenated polymer or similar rigid material so that the wraps 702-1, 702-2 may stand up and retain a shape that allows for the wraps 702-1, 702-2 to form a well into which the therapeutic fluid 704 may be poured and retained without the sides of the wraps 702-1, 702-2 falling. In one example, the wraps 702-1, 702-2 of FIGS. 7 and 8 may include one or more grommets 804 located towards a top edge of the wraps 702-1, 702-2. In one example, a rope 806 may be fed through the grommets 804 of a first wrap 702-1, wrapped around a back of the horse 406, and coupled to the grommets 804 of a second wrap 702-2 in order to assist in holding up the wraps 702-1, 702-2 and ensure that the wraps 702-1, 702-2 do not fall and spill any of the therapeutic fluid 704 and ensure that the therapeutic fluid 704 is in continuous contact with the forearm 602, cannon (not shown), and fetlock (not shown) of a horse 406. The grommets 804 may be formed on either side of the wraps 702-1, 702-2.

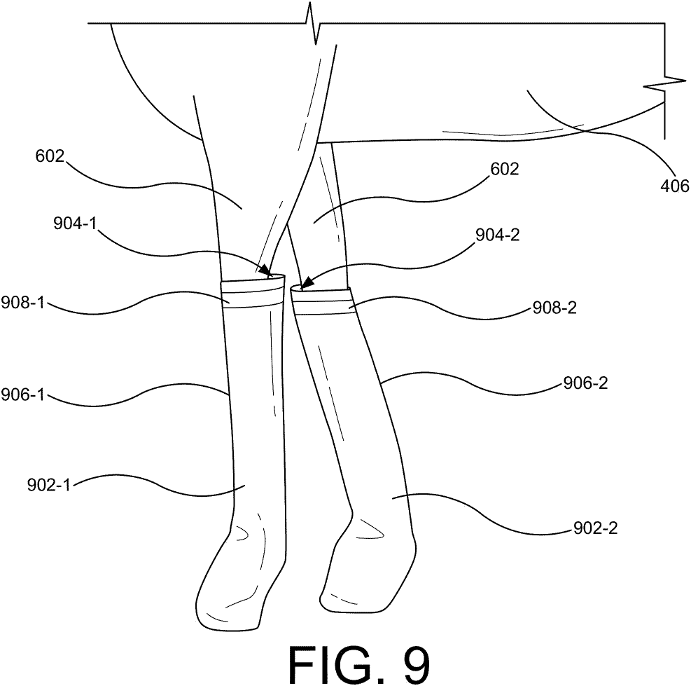

FIG. 9 illustrates a pair of wraps 902-1, 902-2 for use with a therapeutic fluid and for a forearm 602, cannon (not shown), and fetlock (not shown) of a horse 406, according to an example of the principles described herein. The wraps 902-1, 902-2 may include a sock-type form factor including an outer housing 906-1, 906-2 with openings 904-1, 904-2 to allow for the insertion of a therapeutic fluid 704 such as a nano-dimensioned ice crystal slurry. In one example, the wraps 902-1, 902-2 of FIG. 9 may include, for example, a PVC-backed polyester. The wraps 902-1, 902-2 may further include one or more fasteners 908-1, 908-2 to couple the top portion of the wraps 902-1, 902-2 to secure the wraps 902-1, 902-2 to, for example, the forearm 602 of the horse 406. In one example, the fasteners 908-1, 908-2 may include buckles, straps, rope, elastic, and other fastening devices. In one example, the fasteners 908-1, 908-2 may be used to close the openings 904-1, 904-2 to ensure that the therapeutic fluid 704 does not leak outside the wraps 902-1, 902-2. Further, the fasteners 802 may be used to accommodate for the insertion of a body part into the wraps 702-1, 702-2 such as a forearm 602, cannon 402, fetlock 404, and/or hoof of a horse 406. In this manner, the therapeutic fluid may contact the skin of the horse 406 to ensure a direct contact with the therapeutic fluid 704 as the nano-dimensioned ice crystal slurry penetrates the skin and other tissues to deliver a superior therapeutic effect. The wraps 902-1, 902-2 of FIGS. 9 and 8 may be made of a relatively flexible material such as, for example, fabric, a plastic, a rubber, or other bendable material so that the wraps 902-1, 902-2 may generally conform with the shape of the forearm 602, the cannon (not shown), the fetlock (not shown) or other portion of the horse 406 or any other tissues of an animal. The therapeutic fluid 704 may be poured and retained without the wraps 902-1, 902-2 as gravity pulls the therapeutic fluid 704 between the walls of the wraps 902-1, 902-2 and the skin of the animal.

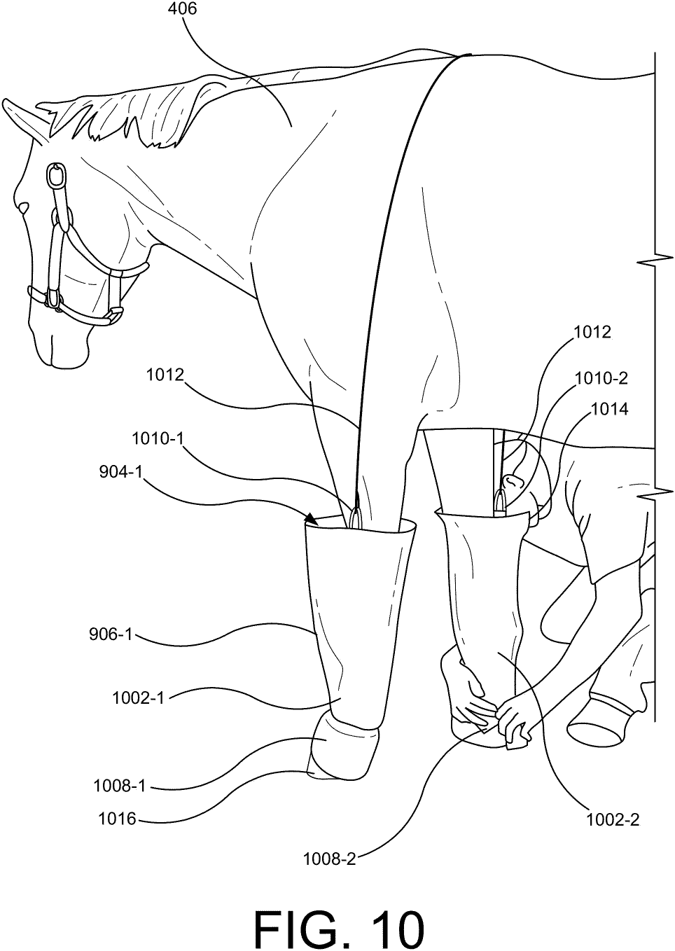

FIG. 10 illustrates a pair of wraps 1002-1, 1002-2 for use with a therapeutic fluid 704 and for a forearm, cannon, and fetlock of a horse 406, according to an example of the principles described herein. In one example, the wraps 1002-1, 1002-2 of FIG. 10 may include, for example, a PVC-backed polyester which provides for flexibility of the material to allow the material to form to the shape of the horse 406 as well as to allow for the therapeutic fluid 704 to fill the space between the wraps 1002-1, 1002-2 and the horse 406 and expand the outer dimensions of the wraps 1002-1, 1002-2 to an extent of the diameter of the wraps 1002-1, 1002-2. In one example, the wraps 1002-1, 1002-2 may be held up with a rope 1012 coupled to one or more loops 1010-1, 1010-2 integrated into the top portions of the wraps 1002-1, 1002-2. The rope 1012 may be slung over the back of the horse 406 and coupled to the loops 1010-1, 1010-2. In this manner, the loops 1010-1, 1010-2 and the rope 1012 assist in holding up the wraps 1002-1, 1002-2 so that the therapeutic fluid 704 may be retained within the wraps 1002-1, 1002-2 without the wraps 1002-1, 1002-2 otherwise falling and spilling the therapeutic fluid 704. In a manner similar to the example of FIG. 8, the wraps 702-1, 702-2 may further include one or more fasteners 802 along a length of the wraps 1002-1, 1002-2 may include, for example, one or more zippers, hook-and-loop fasteners, or other fasteners coupled to two edges of a horizontal opening 1014. In one example, the zippers, hook-and-loop fasteners, or other fasteners may be waterproof to ensure that the therapeutic fluid 704 that is introduced into eh interior space of the wraps 1002-1, 1002-2 does not leak outside the wraps 1002-1, 1002-2. Further, the fasteners may be used to accommodate for the insertion of a body part into the wraps 1002-1, 1002-2 such as a forearm 602, cannon 402, fetlock 404, and/or hoof of a horse 406 via the opening 1014. In this manner, the wraps 1002-1, 1002-2 may cause the therapeutic fluid to contact the skin of the horse 406 to ensure a direct contact with the therapeutic fluid 704 as the nano-dimensioned ice crystal slurry penetrates the skin and other tissues to deliver the superior therapeutic effect.

In one example, the wraps 1002-1, 1002-2 may further include a reinforced hoof bed 1016 secured to the bottom of the wraps 1002-1, 1002-2. The reinforced hoof bed may include a non-slip nylon tread or other type of material that may assist in the stabilization and footing of the horse 406 as the horse 406 moves around. Further, the wraps 1002-1, 1002-2 may be secured to the hoof area of the horse 406 using one or more layers of hoof tape or other securing devices that will serve to se3cure the bottom of the wraps 1002-1, 1002-2 to the legs of the horse 406 such that the horse 406 cannot kick off the wraps 1002-1, 1002-2 or otherwise remove the wraps 1002-1, 1002-2.

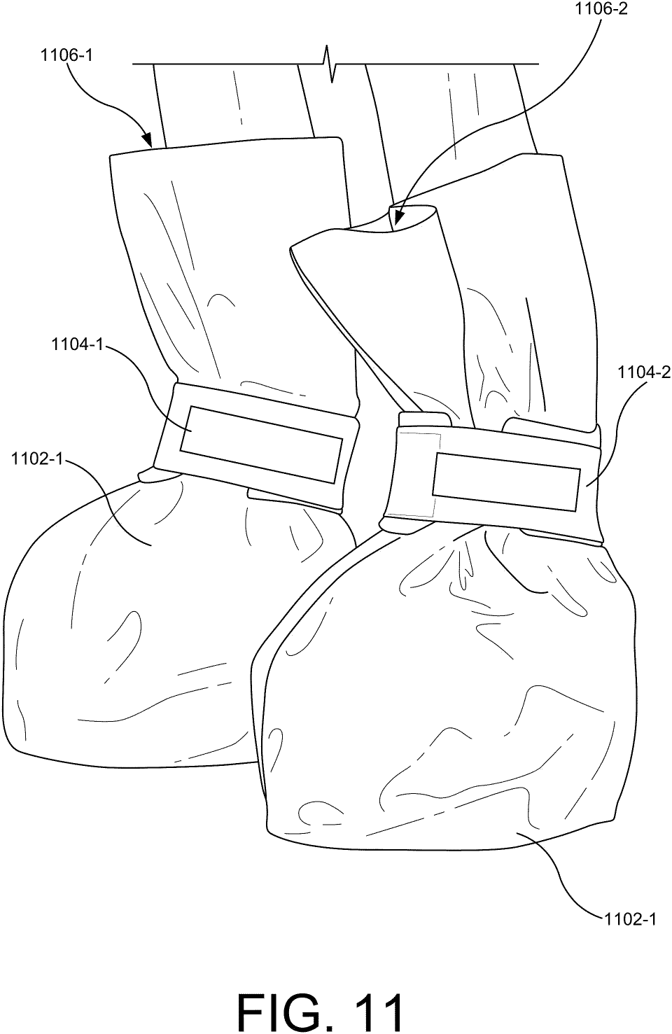

FIG. 11 illustrates a pair of wraps 1102-1, 1102-2 for use with a therapeutic fluid 704 and for a fetlock, a pastern, a heel, and/or a hoof of a horse, according to an example of the principles described herein. The example wraps 1102-1, 1102-2 of FIG. 11 may be shorter in length compared to the examples of, for example, FIGS. 4-10, and may be dimensioned or sized to treat the lower portions of the legs of the horse 406 such as the fetlock, the pastern, the heel, and/or the hoof. The wraps 1102-1, 1102-2 may include one or more straps 1104-1, 1104-2 located just above the hooves to allow for the wraps 1102-1, 1102-2 to be secured around the pasterns of the horse 406 that has a relatively smaller diameter as compared to the hooves such that the straps 1104-1, 1104-2, being coupled to the wraps 1102-1, 1102-2, hold the wraps 1102-1, 1102-2 onto the legs of the horse 406. Once coupled to the horse 406, the wraps 1102-1, 1102-2 may be filled with the therapeutic fluid 704. In one example, the wraps 1102-1, 1102-2 may be made of, for example, a PVC-backed polyester or other water-tight material to allow for the therapeutic fluid 704 to be retained within the wraps 1102-1, 1102-2 and directly contact the skin of the horse 406.

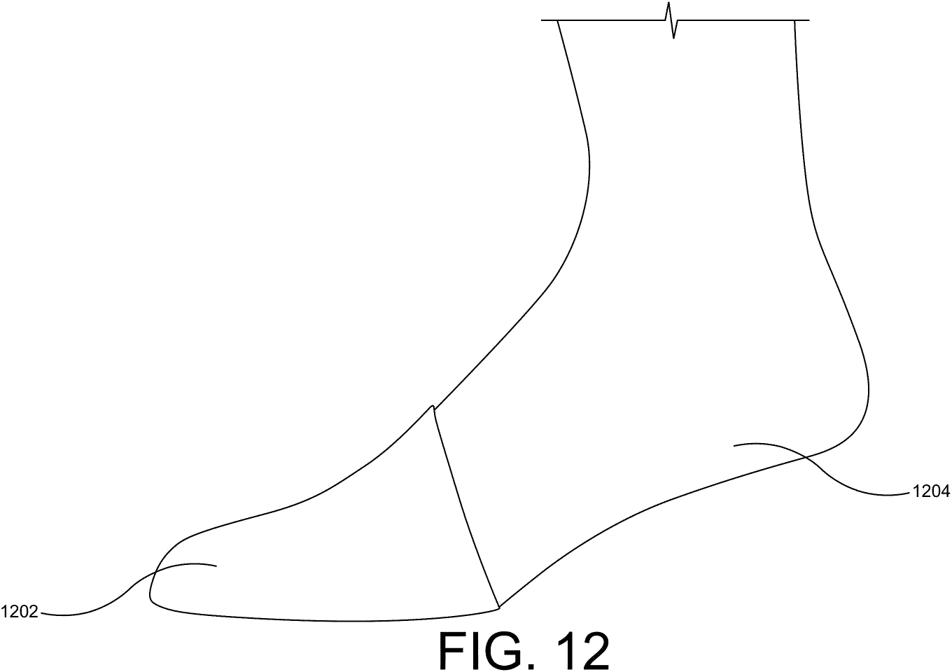

FIG. 12 illustrates a protective cover 1202 for use with a therapeutic fluid and for a foot 1204 of a human, according to an example of the principles described herein. The protective cover 1202 may be made of, for example, a Neoprene® or similar material that may ensure that the toes of the human foot 1204 are protected during a therapy session related to the foot 1204. In this example, the protective cover 1202 may serve to keep the therapeutic fluid 704 away from the toes of the foot 1204 or at least provide some level of thermal protection form the toes of the foot 1204. In one example, it may be beneficial during icing therapy session that are relatively longer than others where the toes, being extremities that may disproportionately suffer a decrease in temperature through longer periods of time. Similar protective covers 1202 may be employed when providing therapies to other portions or areas of a body of an animal in order to isolate those potions or areas from the therapy or unintended drops in temperature.

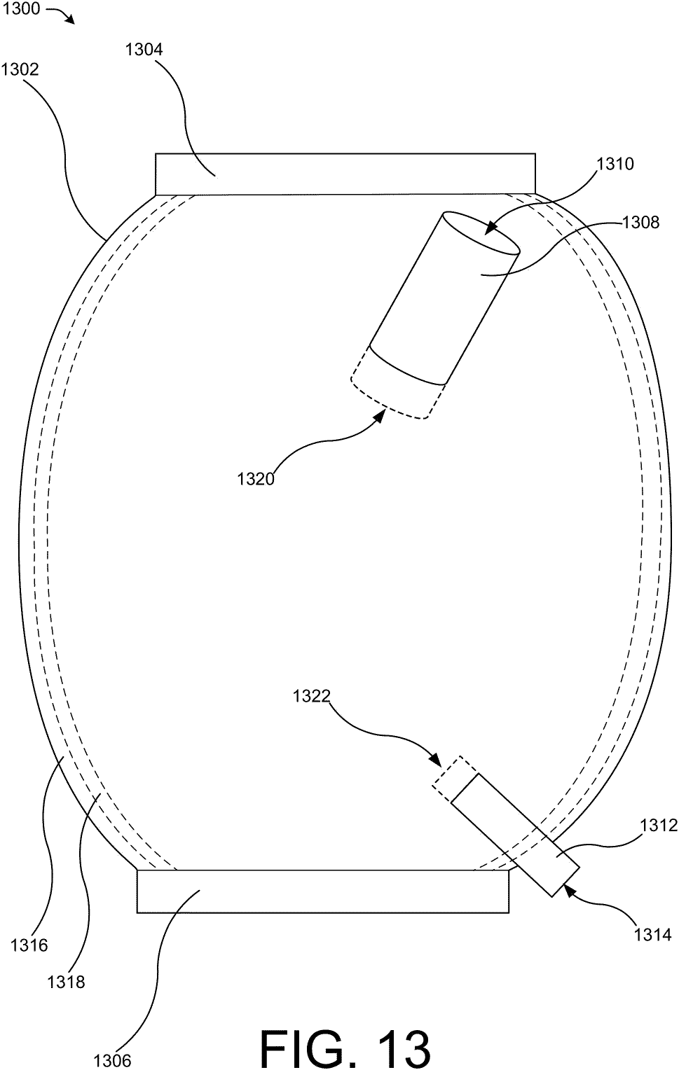

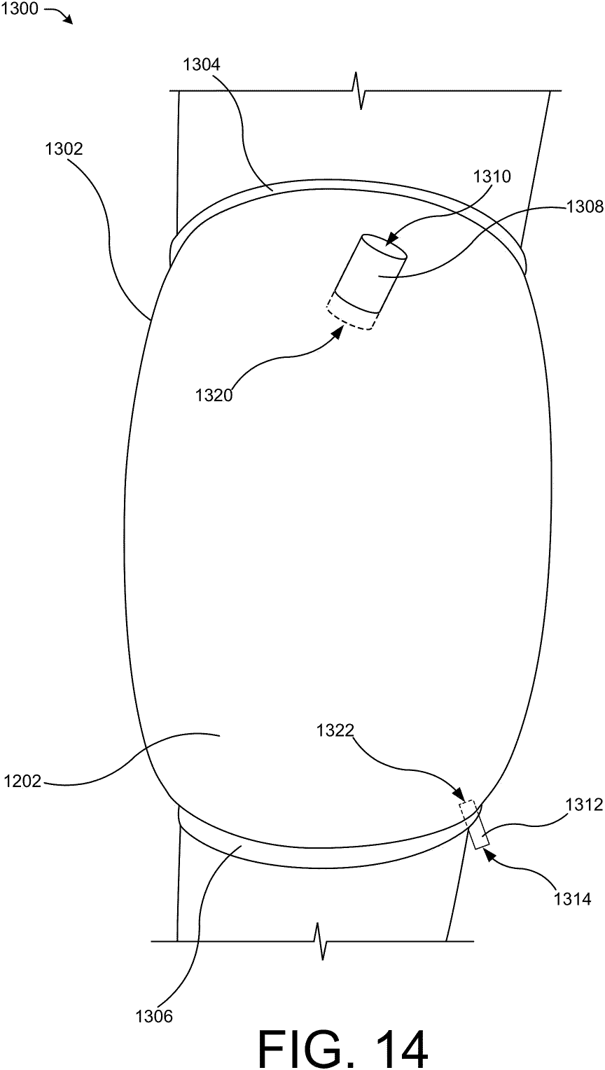

FIGS. 13 and 14 illustrate a wrap 1300 for use with a therapeutic fluid 704 and for the knee area of a human, according to an example of the principles described herein. FIG. 13 depicts the wrap 1300 from a side of the wrap 1300, and FIG. 14 depicts the wrap 1300 from a front view of the wrap 1300. The wrap 1300 may include a shell 1302. In one example, the shell 1302 may wrap around an entirety of the knee of the user. In one example, the shell 1302 may include a reservoir 1318 covered by an outer shell 1316. In one example, the reservoir 1318 may include a three-dimensional (3D) printed plastic to fit over the knee of the user. In one example, the outer shell 1316 may include a Neoprene® or other insulating material to prevent the therapeutic fluid 704 from melting. Further, the outer shell 1316 may cause an outer surface of the reservoir 1318 to not be exposed to the user such that by separating the chilled outer surface of the reservoir 1318.

The wrap 1300 of FIGS. 13 and 14 may further include an inlet port 1308. In one example, the inlet port 1308 may be made of a tube including an inlet 1310 outside of the shell 1302 and an outlet 1320 interior to the shell 1302. In this manner, the therapeutic fluid 704 may be inserted into the reservoir 1318 via the inlet port 1308. In one example, the inlet port 1308 may be located towards the top of the wrap 1300 such that gravity may retain the therapeutic fluid 704 within the shell 1302 during a therapy session. In one example, a plug, a cap, a valve, a stopcock, or other fluid retention device may be applied to the inlet 1310 to retain any therapeutic fluid 704 in the shell 1302.

The wrap 1300 of FIGS. 13 and 14 may further include an outlet port 1312. In one example, the outlet port 1312 may be made of a tube including an inlet 1322 inside the shell 1302 and an outlet 1314 exterior to the shell 1302. In this manner, the therapeutic fluid 704 may be drained from the reservoir 1318 via the outlet port 1312. In one example, the outlet port 1312 may be located towards the bottom of the wrap 1300 such that gravity may assist in the therapeutic fluid 704 within the shell 1302 being drained after a therapy session is completed. In one example, a plug, cap, valve, stopcock, or other fluid retention device may be applied to the outlet 1314 to retain any therapeutic fluid 704 in the shell 1302. Removal or activation of the plug, cap, valve, stopcock, or other fluid retention device may cause the therapeutic fluid 704 to drain from the shell 1302.

The wrap 1300 of FIGS. 13 and 14 may further include a top pressure band 1304 and a bottom pressure band 1306. In one example, the top pressure band 1304 and the bottom pressure band 1306 may be coupled to the shell 1302 including the outer shell 1316 and/or the reservoir 1318. In one example, the top pressure band 1304 and the bottom pressure band 1306 may be made of an elastic material that is capable of applying pressure against the legs of the human and retaining the therapeutic fluid 704 within the shell 1302. In one example, an adhesive capable of at least temporarily sealing the top pressure band 1304 and the bottom pressure band 1306 to the skin of the user and/or adhering the top pressure band 1304 and the bottom pressure band 1306 to the skin of the user. In this manner, the therapeutic fluid 704 may be retained within the shell 1302 of the wrap 1300 throughout the duration of the therapeutic session.

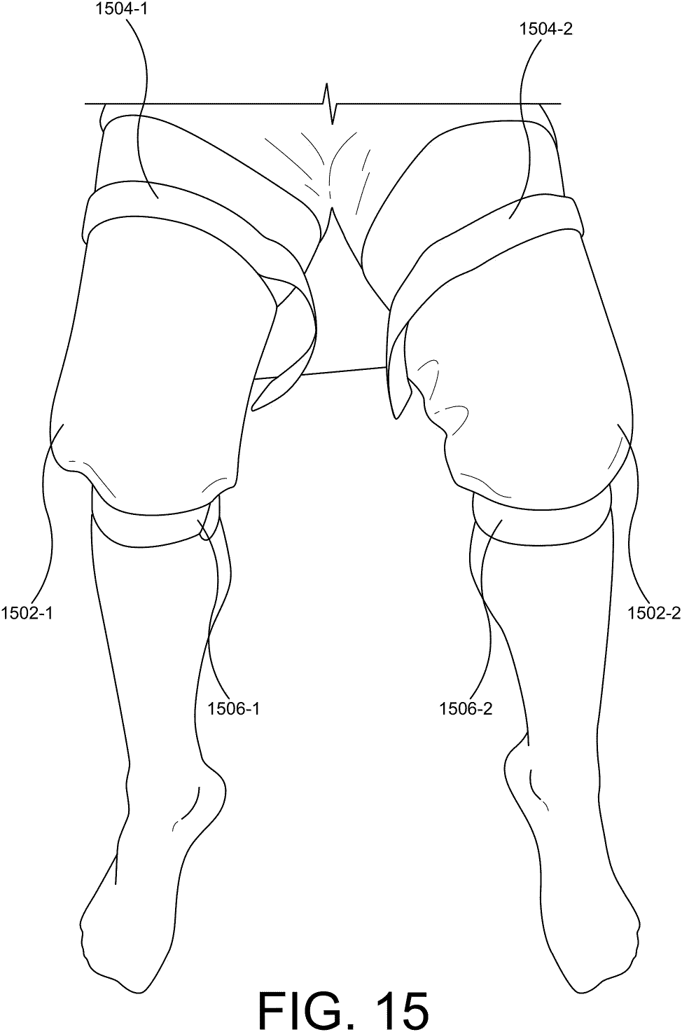

FIG. 15 illustrates a pair of wraps 1502-1, 1502-2 for use with a therapeutic fluid 704 and for the knee area of a human, according to an example of the principles described herein. The example of FIG. 15 depicts the use of the wraps 1502-1, 1502-2 as applied to the knees of a user, but the wraps 1502-1, 1502-2, like other examples described herein, may be applied to one or more different body parts of an animal.

In one example, the wraps 1502-1, 1502-2 may include a layer of Neoprene® with a backing layer made of, for example, nylon. In one example, the wraps 1502-1, 1502-2 may have a thickness of approximately 4.3 millimeters (mm). The wraps 1502-1, 1502-2 may include top straps 1504-1, 1504-2 and bottom straps 1506-1, 1506-2 to secure the wraps 1502-1, 1502-2 to the individual’s body. In one example, the top straps 1504-1, 1504-2 and bottom straps 1506-1, 1506-2 may include a hook-and-loop cinch strap. Further, in one example, an adhesive or other substance capable of at least temporarily sealing the top straps 1504-1, 1504-2 and the bottom straps 1506-1, 1506-2 to the skin of the user and/or adhering the top straps 1504-1, 1504-2 and the bottom straps 1506-1, 1506-2 to the tissues to be treated (e.g., skin of the user) may be used.

In use, the wraps 1502-1, 1502-2 may be positioned at appropriate positions around the knees of the user, and the bottom straps 1506-1, 1506-2 may be sealed against the skin of the user. The therapeutic fluid 704 may be poured into the wraps 1502-1, 1502-2 via the open top straps 1504-1, 1504-2. Once a desired amount of the therapeutic fluid 704 is poured, the top straps 1504-1, 1504-2 may be sealed against the skin of the user. In this manner, direct contact between the therapeutic fluid 704 and the skin of the user may be achieved. Once a therapeutic session has been completed, the top straps 1504-1, 1504-2 and/or bottom straps 1506-1, 1506-2 may be released from the skin of the user allowing the therapeutic fluid 704 to exit the wraps 1502-1, 1502-2 and allowing the user to remove the wraps 1502-1, 1502-2 from their knees.

The above examples of wraps described in connection with FIGS. 1 through 15 include one or more differing aspects, elements, and details which may be used in combination with one another with regard to both their parts and in total. Further, any number of wraps may be used in combination with one another. The wraps of FIGS. 1 through 15 ensure that the therapeutic fluid 704 is continually and directly in contact with the skin of the animal. The therapeutic fluid 704, being a nano-dimensioned ice crystal slurry is able to penetrate the skin of the animal. The therapeutic fluid 704 produced at this nanoscale also allows for dilation, reduction of swelling, and opening of pores of the user’s skin to allow possible therapeutic agents in the nano-ice, frozen cryogenic fluid, or slurry to pass into the skin topically. Similar effects may be experienced in connection with different types of tissues and organs. These therapeutic agents in the nano-ice, frozen cryogenic fluid or slurry may include, for example, methylsulfonylmethane (MSM), glucosamine, aloe including pure aloe, Epsom salts, trehalose, autologous cultured chondrocytes, cytokines for wound healing (e.g., derma gel, silvasorb, chlorhexidine 2%/4%, steroid creams), botulinum toxin type A, onabotulalinumtoxina (e.g., Botox), baclofen, tizanidine, cyclobenzaprine, iodine preparations (e.g., tincture of iodine, potassium iodide, iodophors), copper preparations (e.g., copper sulfate, copper naphthenate, cuprimyxin), sulfur preparations (e.g., monosulfiram, benzoyl disulfide), phenols (e.g., phenol, thymol), fatty acids and salts (e.g., propionates, undecylenates), organic acids (e.g., benzoic acid, salicylic acids), dyes (e.g., crystal [gentian] violet, carbolfuchsin), hydroxyquinolines (e.g., iodochlorhydroxyquin), nitrofurans (e.g., nitrofuroxine, nitrofurfurylmethyl ether), imidazoles (e.g., miconazole, tioconazole, clotrimazole, econazole, thiabendazole), polyene antibiotics (e.g., amphotericin B, nystatin, pimaricin, candicidin, hachimycin), allylamines (e.g., naftifine, terbinafine), thiocarbamates (e.g., tolnaftate), and miscellaneous agents (e.g., acrisorcin, haloprogin, ciclopirox, olamine, dichlorophen, hexetidine, chlorphenesin, triacetin, polynoxylin, amorolfine, Triclosan, Microban, Iodine, O-phenylphenol, Hydronium, Dakin’s Solution, hydrogen peroxide, honey, vinegar, essential oils, Erythromycin (e.g., antibiotics), mesenchymal stem cells (e.g., MSCs), platelet-rich plasma (PRP), autologous conditioned serum (ACS) and autologous protein solution (APS), chlorhexidine, dermatophilus congolensis, and combinations thereof, among other chemical compositions.

In one example, the cryogenic fluid composition may be formulated within an auger system to allow for one or more formations including dendrites, plates, solid prisms, hollow prisms, solid columns, hollow columns, and needles, among other formations. In one example, the formations may be generated along an interior wall of a core of the auger system at between approximately 0° to -5° C. range (approximately 32° F. to 23° F.). At this range of temperatures, the formations may be scraped or knocked off the interior wall. In one example, the cryogenic fluid composition may be formulated as a supersaturation in grams per meter cubed (g/m3) at approximately 0 to 0.3 g/m3. The formation of the cryogenic fluid or slurry at these temperatures and supersaturation levels allows for the formulations described herein to form rather than, for example, relatively larger formulations. As the formations are scraped or knocked off the interior wall, the formations may be subjected to shear forces that create even smaller formations such as the nano-ice formations described herein. Thus, in the first instance of creation, the formations may be relatively smaller, and the formations further decrease in size as they are scraped or knocked off the interior wall.

### Examples of Patch Devices

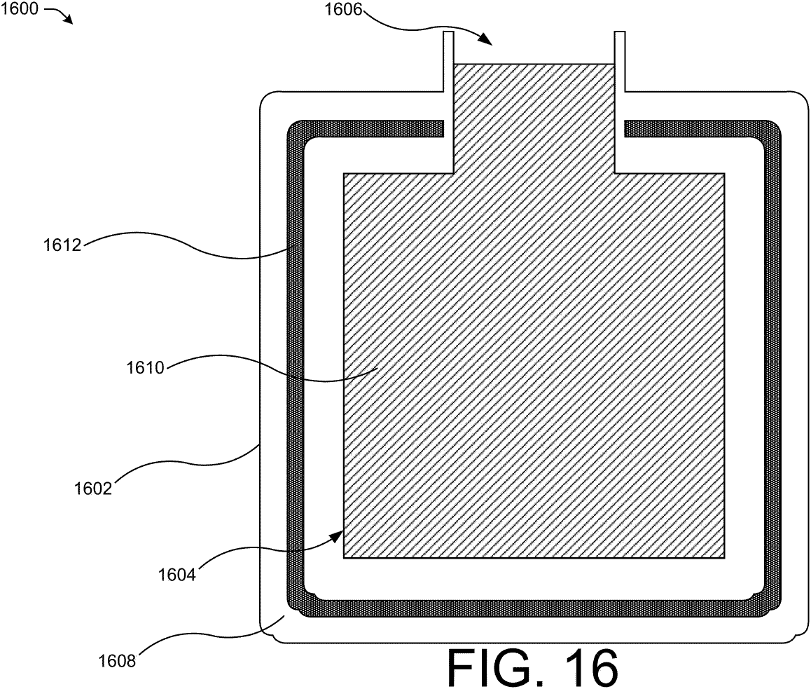

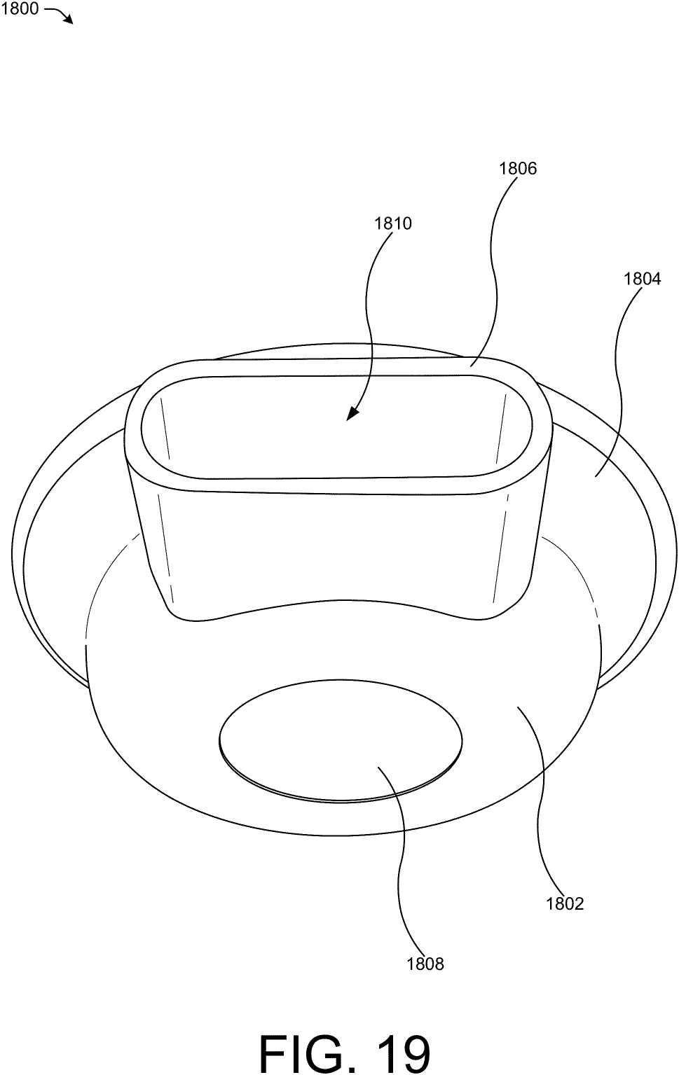

Having described one or more examples of the wraps depicted in FIGS. 1 through 15, one or more examples of patches will now be described in connection with FIGS. 16 through 40. The patch may include a substrate, a seal to couple the substrate to a surface of an animal to be treated, and at least one port defined in the substrate through which a therapeutic fluid may be introduced between the surface to be treated and the substrate such that the therapeutic fluid directly contact the surface of the animal. In the examples of the wraps depicted in FIGS. 1 through 15, the wraps may be made of a relatively more flexible and/or elastic material that make take a shape of the part of the animal around which the wrap is coupled. In contrast, although the patches described herein in connection with FIGS. 16 through 40 may be made of an elastic material, the patches may have a relatively lower degree of flexibility and/or elasticity but are still able to at least a degree to conform to a surface to be treated.

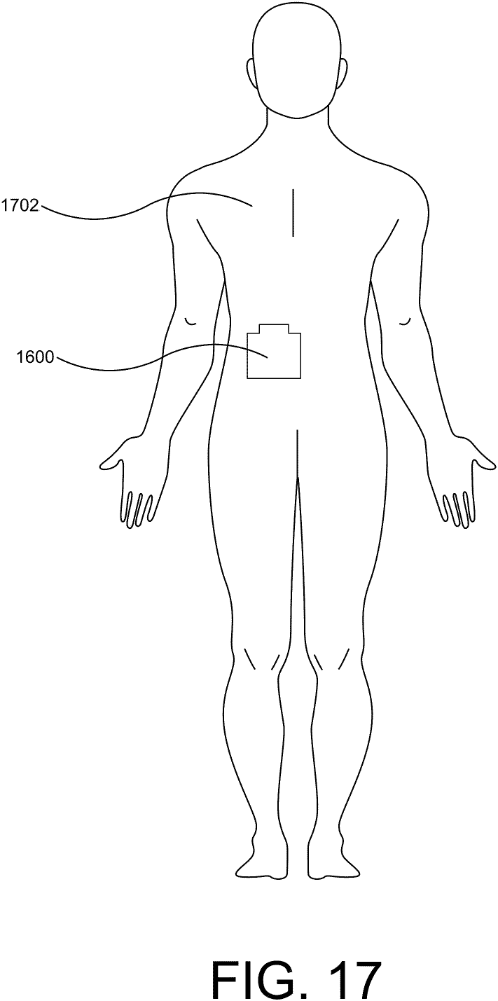

Turning again the figures, FIG. 16 illustrates a patch 1600 for use with a therapeutic fluid, according to an example of the principles described herein. FIG. 17 illustrates the patch 1600 for use with a therapeutic fluid and for the application to an area of a human body 1702, according to an example of the principles described herein. The example of FIG. 16 may depict a generic version of the examples of the patches of FIGS. 16 through 40. The example of FIG. 16 may include a shell 1602. A cavity 1604 may be defined in a first side 1608 of the shell 1602. As described in more detail herein, the cavity 1604 may be filled with the therapeutic fluid 1610. Further, the first side 1608 of the shell 1602 is the side of the shell 1602 that abuts the tissues of the animal that are to be therapeutically treated.

The patch 1600 may include an adhesive 1612 applied to the first side 1608 of the shell 1602 along an outer perimeter of the cavity 1604. The adhesive may include any substance capable of at least at least temporarily sealing the first side 1608 of the patch 1600 to the tissues of the animal to be treated (e.g., the skin of a user or the skin/hair of a horse). The adhesive 1612 may serve to both couple the patch 1600 to the animal as well as to create a seal such that when the therapeutic fluid 1610 is introduced into the cavity 1604, the adhesive 1612 seals the therapeutic fluid 1610 within the cavity 1604 along portion of the first side 1608 where the adhesive 1612 is applied.

The patch 1600 may further include an inlet 1606 fluidically coupled to the cavity 1604 and through which the therapeutic fluid 1610 may be poured into the cavity 1604. In one example, the inlet 1606 may be placed at a top portion of the patch 1600 such that gravity continually pulls the therapeutic fluid 1610 downward and retains the therapeutic fluid 1610 within the cavity 1604. In one example, the patch 1600 may further include a lid, a plug, a cap, a valve, a stopcock, or other fluid retention device may be applied to the inlet 1606 to ensure that the therapeutic fluid 1610 does not leak out the inlet 1606. Further, in one example, the patch 1600 may include an outlet through which the therapeutic fluid 1610 may be removed from the space created between the cavity 1604 and the tissues of the animal to which the patch 1600 is coupled. More details regarding the inlet 1606 and outlet are described herein in connection with other examples.

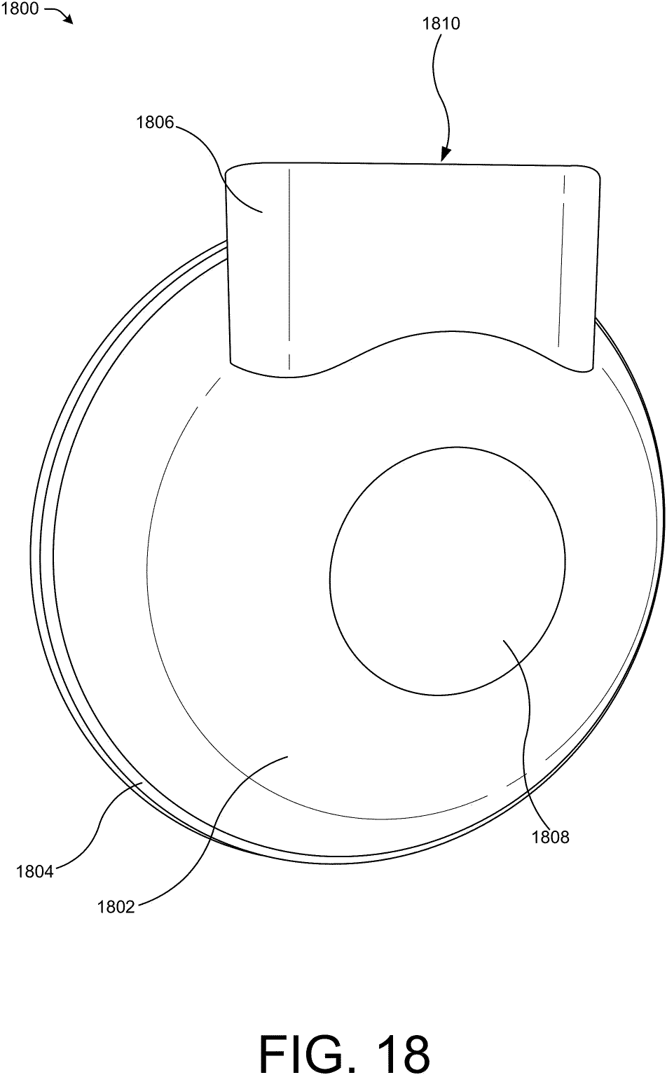

The example described in connection with the patch 1600 of FIG. 16 may apply to the examples described in connection with FIGS. 18 through 40. For example, the application of an adhesive serves to seal the therapeutic fluid 1610 within the example patches of FIGS. 18 through 40. Further, each examples of patches of FIGS. 18 through 40 may include a cavity in which the therapeutic fluid 1610 may be stored and directly contact the tissues of the animal.

Further, as depicted in FIG. 17, the patch 1600 may be applied to any portion of a human body 1702 such as, for example, a lower back area of the human body 1702. The patch 1600 may take any form or size to allow for the patch 1600 to be applied to any area of the human body 1702 including areas of the human body 1702 with relatively smaller surface areas such as hands, feet, arms, etc. Still further, the patch 1600 may be applied to animals other than humans such as, for example, horses as depicted herein. However, the systems and methods described herein may be applied to any tissues of any animal.

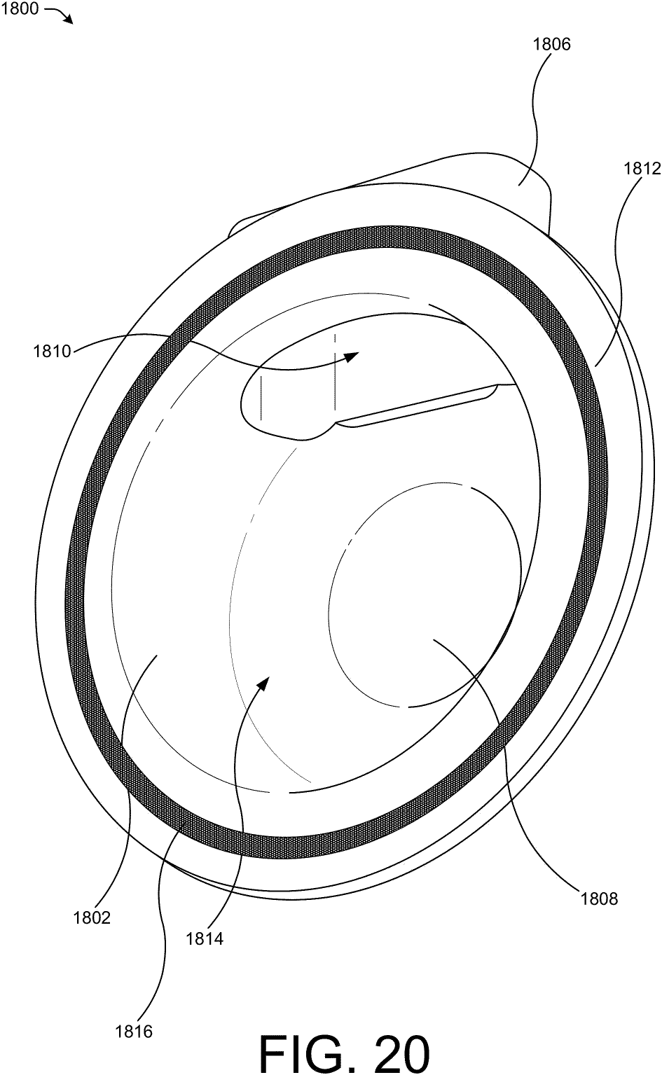

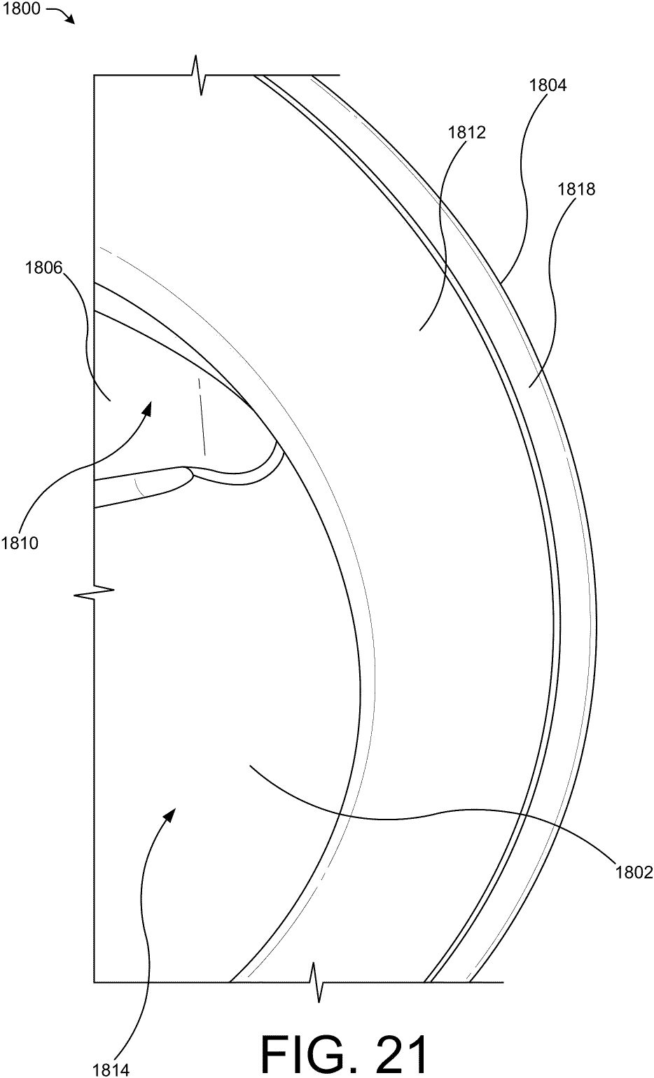

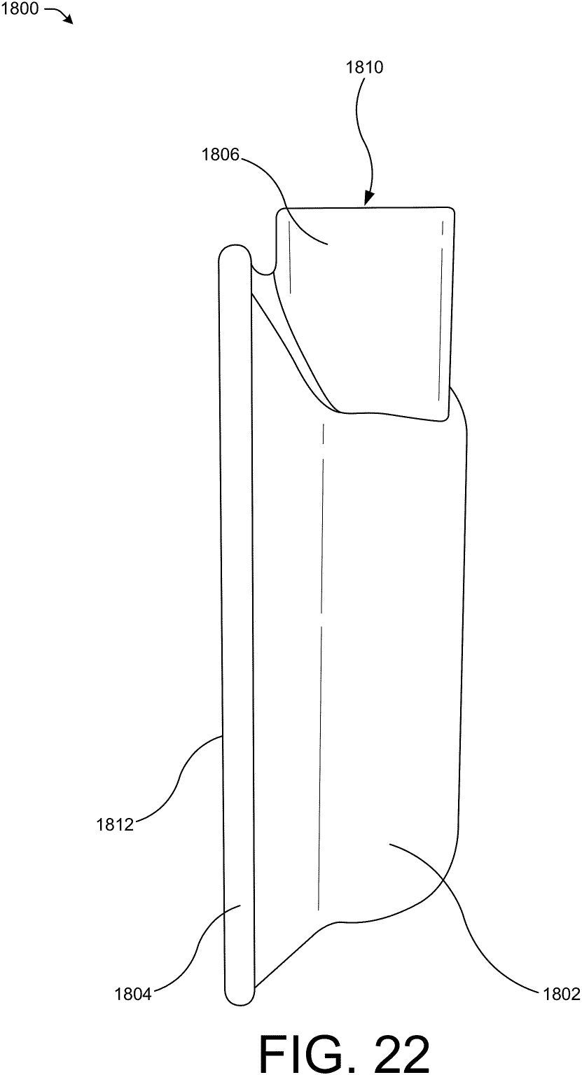

FIG. 18 illustrates a front perspective view of a patch 1800, according to an example of the principles described herein. FIG. 18 illustrates a top perspective view of the patch 1800 of FIG. 18, according to an example of the principles described herein. FIG. 20 illustrates a rear perspective view of the patch 1800 of FIG. 18, according to an example of the principles described herein. FIG. 21 illustrates a close-up, rear perspective view of the patch 1800 of FIG. 18, according to an example of the principles described herein. FIG. 22 illustrates a side view of the patch 1800 of FIG. 18, according to an example of the principles described herein. The patch 1800 may include a dome 1802 coupled to a flange 1804. In one example, the dome 1802 and the flange 1804 may be monolithically formed. An inlet 1806 may be coupled to or monolithically formed with the dome 1802 and/or the flange 1804. The inlet 1806 defines a via 1810 that is fluidically coupled to a cavity 1814 defined by the dome 1802. The dome 1802 serves as a reservoir that holds a volume of the therapeutic fluid 1610.

In one example, the dome 1802, the flange 1804, and/or the inlet 1806 may be made of a rubber or plastic material. For example, the dome 1802 and the flange 1804 may be made of thermoplastic polyurethane (TPU), acrylonitrile butadiene styrene (ABS), polylactic acid (PLA), polyethylene terephthalate glycol (PETG), polyethylene terephtathalate (PET), high-impact polystyrene (HIPS), thermoplastic polyurethane (TPU) and aliphatic polyamides (nylon), natural rubber, styrene-butadiene rubber (SBR), uncured rubber, volcanized rubber, butyl (IIR), nitrile (NBR), Neoprene® (CR), ethylene propylene diene monomer (EPDM), silicone (Q), Viton® (FKM), polyurethane (AU), hydrogenated nitrile (HNBR), other plastics and rubbers, and combinations thereof. The material the dome 1802, the flange 1804, and/or the inlet 1806 may be flexible to fit to contours of portions of a body of an animal.

An indicator 1808 may be formed with or embedded within the dome 1802. The indicator 1808 may be any device capable of providing any form or type of information to a user. In one example, the indicator 1808 may include a thermoplastic indicator that may change color based on the temperature within the dome 1802 as the therapeutic fluid 1610 is poured into the cavity 1814. In one example, the indicator 1808 may include a window through which a user or administrator may view the therapeutic fluid 1610 within the cavity 1814 of the dome 1802. In one example, the indicator 1808 may include an user interface including a display device, one or more sensors, and/or actuators that serve to present information to the user or administrator such as a temperature of the therapeutic fluid 1610 within the cavity 1814, a temperature within the cavity 1814 of the dome 1802, and a timer indicating a duration of time such as a duration of a therapy session, among other environmental characteristics associated with the use of the patch 1800 that may be sensed by the sensors and displayed on the display device. Further, the indicator 1808 may include one or more actuators that may be used to provide information to the user or provide additional types of therapy. For example, the actuator(s) of the indicator 1808 may include a haptic device such as a vibrating motor to indicate to a user or administrator a duration of or completion of a therapeutic session and/or provide additional tissue stimulation. The actuator may further include a speaker device capable of making noises such as beeps to notify a user or administrator. The indicator 1808 may be located anywhere within or on the patch 1800. The indicator 1808 may also display a pressure or compression within the patch 1800. Further, the actuators may include devices that cause the pressure or compression within the patch 1800 to be changed.

In a manner similarly described above in connection with the wraps of FIGS. 1 through 15, an adhesive 1816 may be applied to a first side 1812 of the flange 1804 to which the patch 1800 will interface with the tissues of the animal. The adhesive 1816 serves to couple, at least temporarily, the patch 1800 to the tissues of the animal and create a fluid tight seal between the dome 1802 and/or flange 1804 and the outer portions of the tissues of the animal. In this manner, the therapeutic fluid 1610 may be contained against the tissues of the animal. In one example, the adhesive 1816 may be placed in a dam 1818 defined in the first side 1812 of the flange 1804. However, the adhesive 1816 may be placed anywhere along the first side 1812 of the flange 1804.

In one example, a suction force may be used to couple the patch 1800 to the tissues of the animal. In this example, the therapeutic fluid 1610 may be placed within the cavity 1814, pressure may be applied to the dome 1802 to remove extraneous air from the cavity 1814 and/or the via 1810 of the inlet 1806, and the inlet 1806 may be sealed. Once the deformation of the dome 1802 is removed and allowed to return to its pre-deformed state, a negative pressure is created within the cavity 1814 that causes the patch 1800 to suction to the tissues of the animal. In one example, this suction effect may be used in conjunction with the adhesive 1816. In one example, the suction effect may be removed by unstopping or unsealing the via 1810 of the inlet 1806 and allowing air to enter the cavity 1814 and removing the negative pressure within the cavity 1814. In one example, the suction force may be released with air valves, flaps, or tabs to break the suction. This suction method may be employed in any of the examples of FIGS. 16 through 40 and may be used with or without the adhesives described herein in the examples of FIGS. 16 through 40. Thus, a seal may include a suction device and/or effect that creates negative fluid pressure between the patch and the tissues of an animal to create a partial vacuum between the patch and the tissues of an animal to be treated.

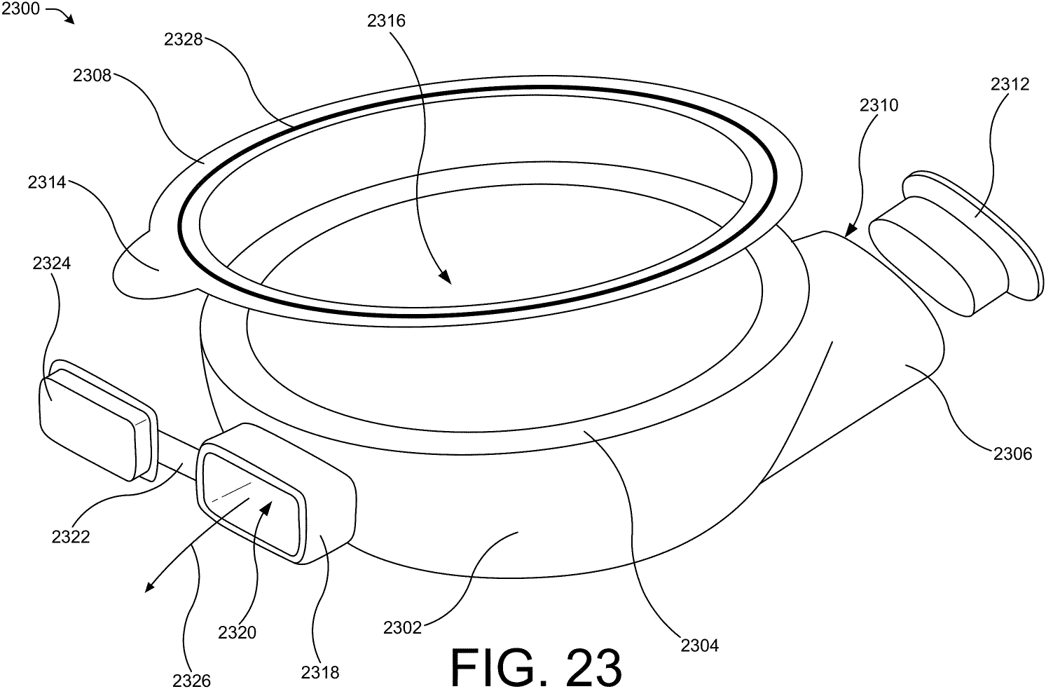

FIG. 23 illustrates an exploded view of a patch 2300, according to an example of the principles described herein. The patch 2300 may include a dome 2302 including a circular base 2304. An inlet 2306 may be coupled to or monolithically formed with the dome 2302 and/or the circular base 2304. The inlet 2310 defines a first via 2310 that is fluidically coupled to a cavity 2316 defined by the dome 2302. The patch 2300 may further include an outlet 2318 coupled to or monolithically formed with the dome 2302. The outlet 2318 defines a second via 2320 that is fluidically coupled to the cavity 2316 defined by the dome 2302.

In one example, the dome 2302, the circular base 2304, the inlet 2306, and/or the outlet 2318 may be monolithically formed. In one example, the dome 2302, the circular base 2304, the inlet 2306, and/or the outlet 2318 may be made of a rubber or plastic material. For example, the dome 2302, the circular base 2304, the inlet 2306, and/or the outlet 2318 may be made of thermoplastic polyurethane (TPU), acrylonitrile butadiene styrene (ABS), polylactic acid (PLA), polyethylene terephthalate glycol (PETG), polyethylene terephtathalate (PET), high-impact polystyrene (HIPS), thermoplastic polyurethane (TPU) and aliphatic polyamides (nylon), natural rubber, styrene-butadiene rubber (SBR), uncured rubber, volcanized rubber, butyl (IIR), nitrile (NBR), Neoprene® (CR), ethylene propylene diene monomer (EPDM), silicone (Q), Viton® (FKM), polyurethane (AU), hydrogenated nitrile (HNBR), other plastics and rubbers, and combinations thereof. The material the dome 2302, the circular base 2304, the inlet 2306, and/or the outlet 2318 may be flexible to fit to contours of portions of a body of an animal.

The patch 2300 may further include a first plug 2312 dimensioned to fit within the inlet 2306 and serve to ensure that any fluid such as the therapeutic fluid 1610 does not leak out of the cavity 2316 of the dome 2302 during a therapy session. Similarly, the outlet 2318 may include a second plug 2324 dimensioned to fit within the outlet 2318 and similarly serve to ensure that any fluid such as the therapeutic fluid 1610 does not leak out of the cavity 2316 of the dome 2302 during the therapy session. In one example, the second plug 2324 may be coupled to an edge of the outlet 2318 via a living hinge 2322 such that the second plug 2324 is not separated from the patch 2300 and become lost. In one example, the first plug 2312 may be similarly coupled to the inlet 2306 via living hinge (not shown). In one example, the first plug 2312 and the second plug 2324 may form an engineering fit with the inlet 2306 and the outlet 2318. As used in the present specification and in the appended claims, the term “engineering fit” is meant to be understood broadly as any clearance between two mating parts such that the mating parts can, at one end of the spectrum, move or rotate independently from each other or, at the other end, are temporarily or permanently joined together. The engineering fit may include, for example, a clearance fit (e.g., one of a loose running fit, a free running fit, a close running fit, a sliding fit, and a location fit), a transition fit (e.g., one of a similar fit, and a fixed fit), and an interference fit (e.g., one of a press fit, a driving fit, and a forced fit). In this manner, the first plug 2312 and the second plug 2324 may hermetically seal the patch 2300 at the inlet 2306 and the outlet 2318 such that the therapeutic fluid 1610 cannot leak out of the cavity 2316. In one example, the first plug 2312 and/or the second plug 2324 may be made of a polymer such as a plastic material or a rubber material.

The patch 2300 may further include a silicone undermounted ring 2308. The ring 2308 may include an adhesive 2328 such as the Silbione™ silicone adhesive manufactured and distributed by Elkem. The adhesive 2328 may be placed along a perimeter of the ring 2308, and may be placed on one or both sides of the ring 2308. In one example, the ring 2308 may be disposable wherein, once removed, the ring 2308 may be thrown away, and replaced by a new ring 2308 such as between applications of the patch 2300. In one example, a removal tab 2314 may be formed on the ring 2308 to assist a user in removing the patch 2300 from the user and/or removing the ring 2308 from the patch 2300.

In use, the patch 2300 may be secured to a portion of the body of an animal by placing the adhesive 2328 on one or both sides of the ring 2308, applying the ring 2308 to the circular base 2304, and applying the patch 2300 to the tissues of the animal with the ring 2308 being interposed between the circular base 2304 and the tissues of the animal. The second via 2320 of the outlet 2318 may be closed by placing the second plug 2324 into the second via 2320. At this point, the cavity 2316 of the patch 2300 is sealed against the tissues of the animal via the ring 2308 and sealed against any therapeutic fluid 1610 exiting the patch 2300 through the second via 2320 as indicated by arrow 2326. In this state, the therapeutic fluid 1610 may be introduced into the cavity 2316 of the patch 2300 through the first via 2310 of the inlet 2306. At this point, the therapeutic fluid 1610 may make direct contact with the tissues of the animal and provide the intended therapy to the animal. In one example, a user or administrator may also engage the first plug 2312 with the inlet 2306 in order to close the first via 2310 and eliminate the possibility of the therapeutic fluid 1610 exiting the cavity 2316 of the patch 2300 through the first via 2310. In this manner, the patch 2300 may be hermetically sealed with the therapeutic fluid 1610 within the cavity 2316.

The therapeutic fluid 1610 may be retained within the patch 2300 for the duration of a therapy session. Once the therapy session is completed, the user or administrator may remove the therapeutic fluid 1610 from the cavity 2316 by removing the second plug 2324 form the second via 2320 of the outlet 2318 and allowing gravity to pull the therapeutic fluid 1610 out of the cavity 2316. In one example, the first plug 2312 may also be removed from the inlet 2306 to allow for air to enter the cavity 2316 and remove any suction forces that may inhibit the flow of the therapeutic fluid 1610 out of the outlet 2318 as indicated by arrow 2326.

After the therapy session is completed and the therapeutic fluid 1610 is removed from the cavity 2316, the patch 2300 may be removed from the surface of the tissues of the animal by a user grasping the removal tab 2314 of the ring 2308 and overcoming the force of the adhesive 2328 to remove the ring 2308 from the tissues of the animal. Further, the ring 2308, being a disposable element in one example, may be removed from the circular base 2304 of the patch 2300 by a user grasping the removal tab 2314 of the ring 2308 and overcoming the force of the adhesive 2328 to remove the ring 2308 from the circular base 2304.

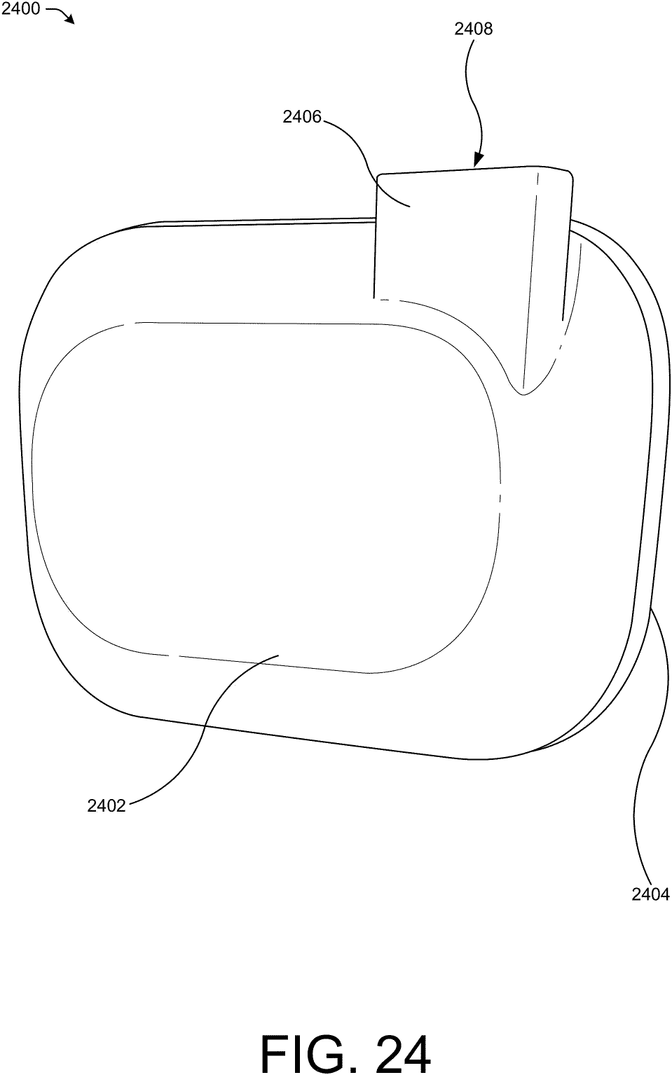

FIG. 24 illustrates a front perspective view of a patch 2400, according to an example of the principles described herein. The patch 2400 of FIG. 24 may include one or more elements of the patches described herein. The patch 2400 may include a generally square shape with a dome 2402, a square base 2404, an inlet 2406, and a via 2408 through which the therapeutic fluid 1610 may be poured as similarly described in connection with other patches herein. The patch 2400 of FIG. 24 is depicted to demonstrate that the patches described herein may take any shape. In one example, the patch may take the shape of a body part of the animal such as shapes of muscles or muscle groups, appendages, etc. Further, the patches described herein may have any size that may allow for more or less of the surface of the tissues of the animal to be treated.

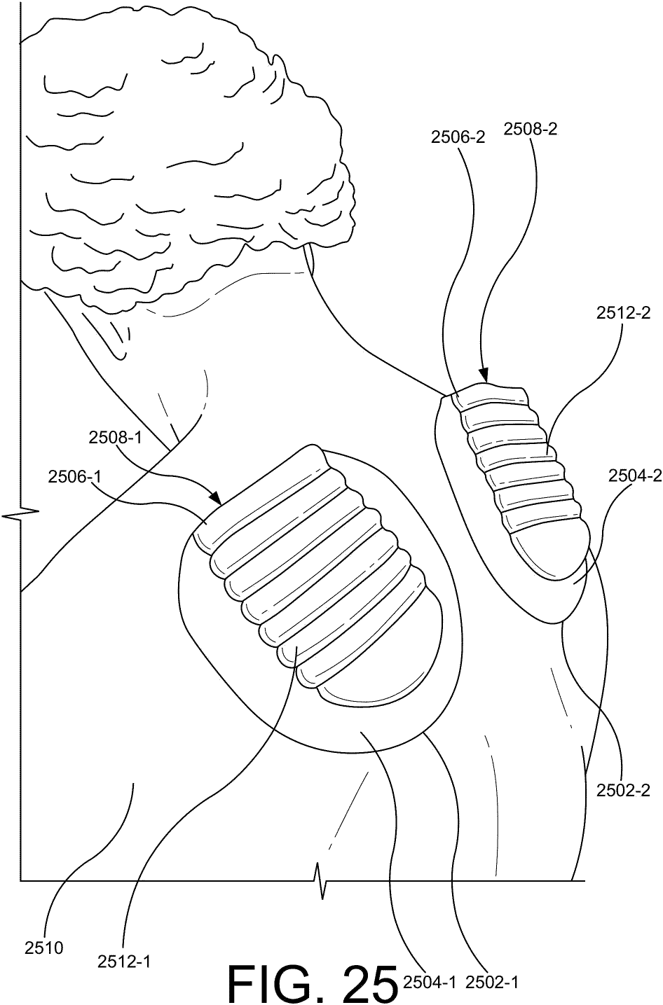

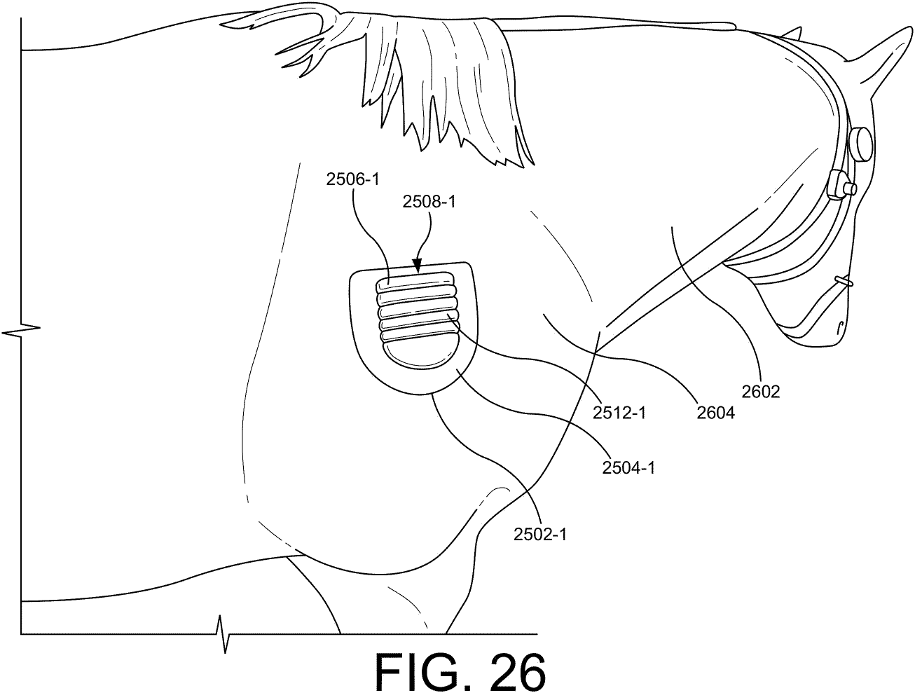

FIG. 25 illustrates a pair of patches 2502-1, 2502-2 (referred to herein as patch(es) 2502) applied to the back of a human 2510, according to an example of the principles described herein. FIG. 26 illustrates the patch 2502-1 (referred to herein as patch(es) 2502) of FIG. 25 as applied to a shoulder 2604 of a horse 2602, according to an example of the principles described herein. The patches 2502 of FIGS. 25 and 26 may include one or more elements of the patches described herein. The patches 2502 may include a generally u-shape with a u-shaped base 2504, an inlet 2506-1, 2506-2, and a via 2508-1, 2508-2 through which the therapeutic fluid 1610 may be poured as similarly described in connection with other patches herein. The patches 2502 of FIGS. 25 and 26 may also include a reservoir 2512-1, 2512-2. In one example, the reservoir 2512-1, 2512-2 may include ribbing or other flexible portions to allow for the patches 2502 to flex around portions of the body of the human 2510 or horse 2602 or other animal. In one example, the patches 2502 of FIGS. 25 and 26 may be made of a liquid silicone rubber to allow for the patches 2502 to provide this flex. Further, in one example, the patches 2502 may be made of a transparent or translucent material such that an amount of therapeutic fluid 1610 within the patches 2502 may be identified by a user or administrator and a sufficient amount of the therapeutic fluid 1610 is at an intended or proper therapeutic amount. Although a cap or other fluid retention device is not depicted in connection with the example of FIGS. 25 and 26, the vias 2508-1, 2508-2 of the inlets 2506-1, 2506-2 may e closed using a fluid retention device in order to ensure that the therapeutic fluid 1610 does not exit the reservoirs 2512-1, 2512-2.

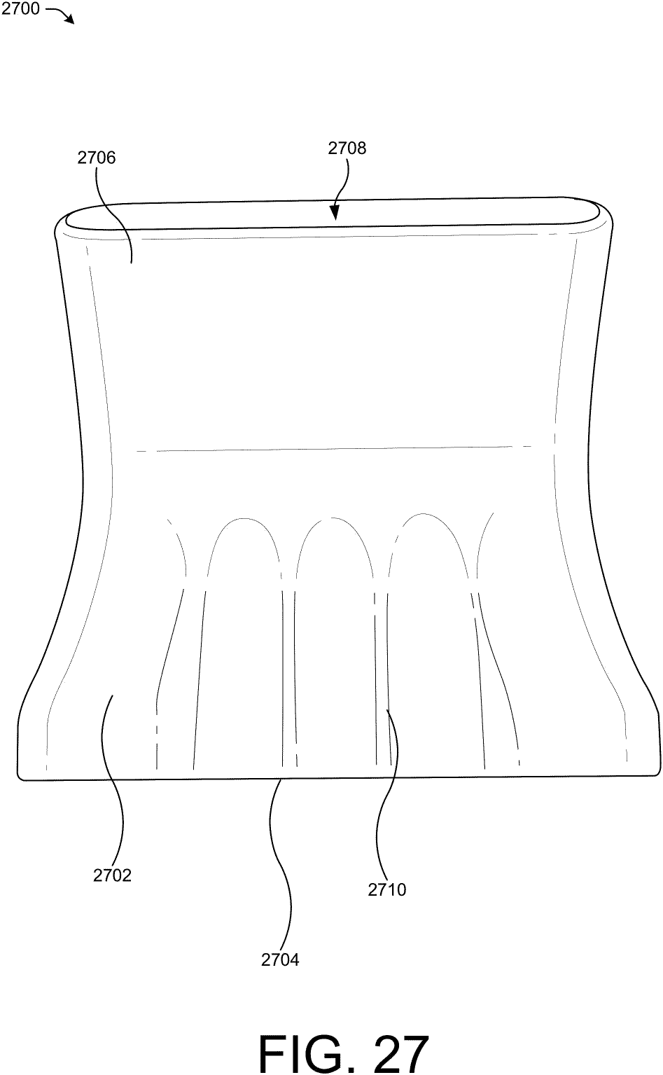

FIG. 27 illustrates a side perspective view of a patch 2700, according to an example of the principles described herein. The patch 2700 may include a reservoir 2702, a base 2704, an inlet 2706, and a via 2708 of the inlet 2706 fluidically coupled to the reservoir 2702. The inlet 2706 allows for a therapeutic fluid 1610 to be poured into the reservoir 2702. The base 2704 may be placed on the tissues of the animal that are to be treated, and the therapeutic fluid 1610 to be poured into the reservoir 2702. In one example, the tissues of the animal may be relatively flat to allow for gravity to pull the therapeutic fluid 1610 into the reservoir 2702. In one example, the reservoir 2702 may include one or more corrugated tubular portions 2710 to all for the patch 2700 to flex against the tissues as well as any movement of the tissues. In one example, an adhesive may be applied to the base 2704 to at least temporarily couple the patch 2700 to the tissues. When adhered to the tissues of the animal via the adhesive, the corrugated tubular portions 2710 may flex with the tissues as the tissues move while still being coupled to the tissues via the adhesive.

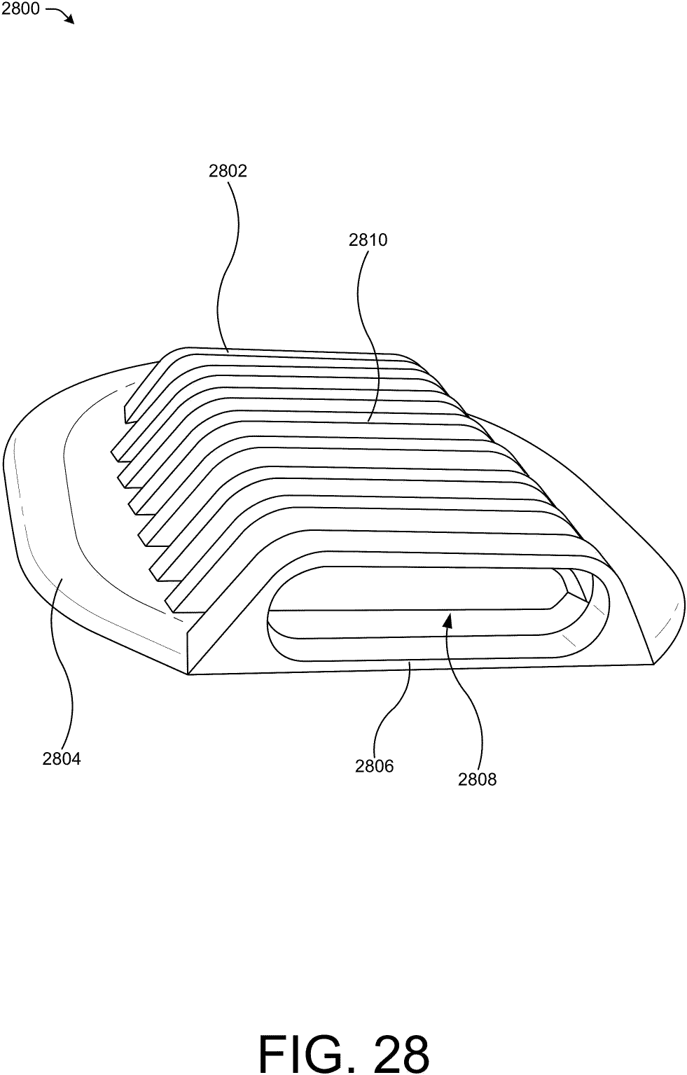

FIG. 28 illustrates a top perspective view of a patch 2800, according to an example of the principles described herein. The patch 2800 of FIG. 28 may include a reservoir 2802, a flange 2804, an inlet 2806, and a via 2808 of the inlet 2806 fluidically coupled to the reservoir 2802. The inlet 2806 allows for a therapeutic fluid 1610 to be poured into the reservoir 2802. The flange 2804 may be placed on the tissues of the animal that are to be treated, and the therapeutic fluid 1610 to be poured into the reservoir 2802. In one example, the reservoir 2802 may include one or more corrugated portions 2810 to all for the patch 2800 to flex against the tissues as well as any movement of the tissues. In one example, an adhesive may be applied to the flange 2804 to at least temporarily couple the patch 2800 to the tissues. When adhered to the tissues of the animal via the adhesive, the corrugated portions 2810 may flex with the tissues as the tissues move while still being coupled to the tissues via the adhesive. In the examples described herein, one or more corrugations, baffles, or accordion type structures may be formed to allow for the flexibility and adaptability for applications over varying skin, hair, and muscle topographies. These corrugations also adapt these examples to skin and muscle movement such that the seal is not comprised or pulled away from the adhered surface.

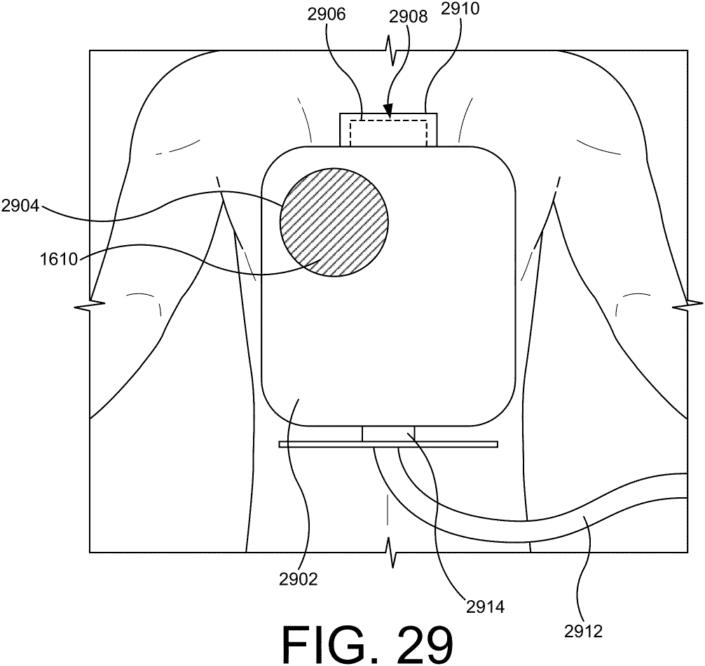

FIG. 29 illustrates a front view of a patch 2902, according to an example of the principles described herein. As mentioned herein, the patches of the examples of FIGS. 16 through 40 may have different sizes. The example patch 2902 of FIG. 29 may be a relatively larger patch 2902 that may be coupled to the tissues of the animal using the techniques described herein. Further, the patch 2902 of FIG. 29 may include a reservoir 2902, an indicator 2904, an inlet 2906, and a via 2908 of the inlet 2806 fluidically coupled to the reservoir 2902. The inlet 2906 allows for a therapeutic fluid 1610 to be poured into the reservoir 2802. The patch 2902 may be placed on the tissues of the animal that are to be treated, and the therapeutic fluid 1610 to be poured into the reservoir 2802.

In the example of FIG. 29, a cap 2910 may be coupled to the inlet 2906 to ensure that any therapeutic fluid 1610 poured into the reservoir 2802 does not spill out of the reservoir 2902. Further, an egress tube 2912 may be coupled to an outlet 2914 to control the amount of therapeutic fluid 1610 within the reservoir 2902.

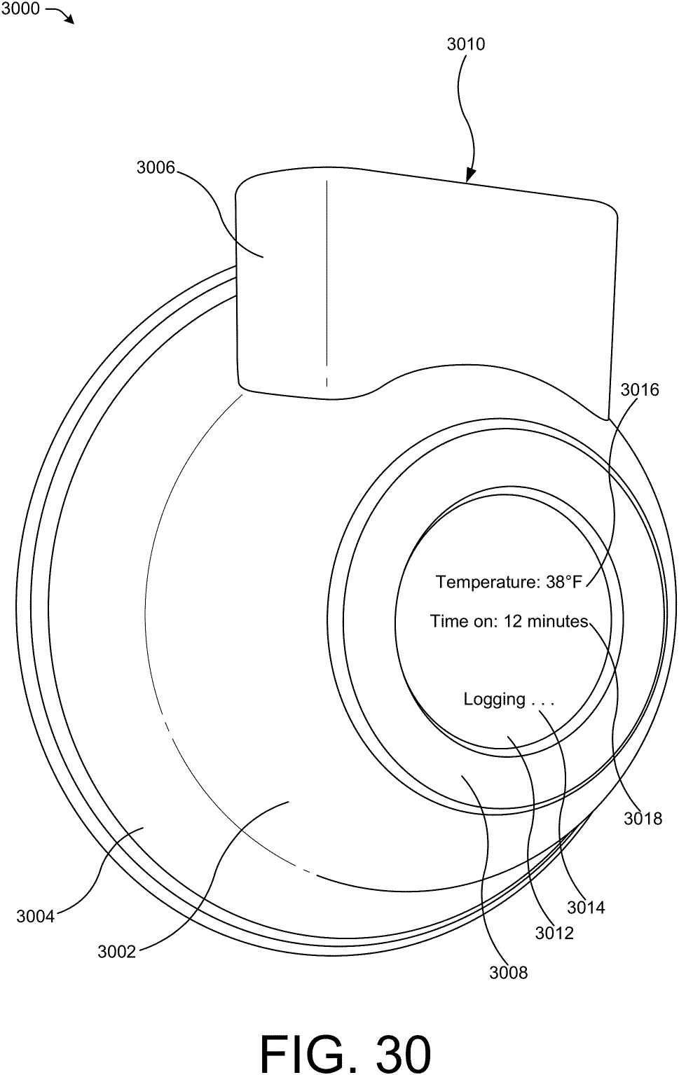

FIG. 30 illustrates a patch 3000 for use with a therapeutic fluid 1610, according to an example of the principles described herein. The patch 3000 may include a dome 3002 coupled to a flange 3004. In one example, the dome 3002 and the flange 3004 may be monolithically formed. An inlet 3006 may be coupled to or monolithically formed with the dome 3002 and/or the flange 3004. The inlet 3006 defines a via 3010 that is fluidically coupled to a cavity defined by the dome 3002. The dome 3002 serves as a reservoir that holds a volume of the therapeutic fluid 1610.

A smart device 3008 may be formed with or embedded within the dome 3002. The smart device 3008 may be any device capable of providing any form or type of information to a user. In one example, the smart device 3008 may include a processing device and memory to process and store data associated with one or more sensors and activators of the smart device 3008.

Further, in one example, the smart device 3008 may include a data transmission device used to send and receive data and/or instructions to and from an associated computing device. The smart device 3008 may employ any type of communication modality such as, for example, Bluetooth®, Wi-Fi, near field communications (NFC) or other modalities to transmit data. The Purpose of transmitting data may include, for example, gathering of data for improving therapeutic use of the patch 3000, device tracking, and other uses.

In one example, data obtained from the sensors may be logged or stored as data within the memory of the smart device 3008. The sensors may include, for example, temperature sensors, sensors that may detect the presence of the therapeutic fluid 1610 within the dome 3002, a clock to identify a duration of time the therapeutic fluid 1610 is within the dome 3002, other types of sensors, and combinations thereof. The smart device 3008 may also display a pressure or compression within the patch 3000. Further, the actuators may include devices that cause the pressure or compression within the patch 3000 to be changed.

The actuators of the smart device 3008 may include a haptic device such as a vibrating motor to indicate to a user or administrator a duration of or completion of a therapeutic session and/or provide additional tissue stimulation.

In one example, the smart device 3008 may include a display device 3012 to convey to a user or administrator information related to the functioning and/or state of the patch 3000. The display device 3012 may include user-readable information such as a current state 3014 of the smart device 3008 (e.g., “Logging ...”), a current temperature 3016, a duration of the therapy session 3018, other types of user-readable information, and combinations thereof. In this manner, a user or administrator may obtain information related to the functioning and/or state of the patch 3000. In one example, the display device 3012 may include a transparent or semi-transparent display device through which a user or administrator may visually inspect the interior of the dome 3002 and determine whether the therapeutic fluid 1610 is inside the dome 3002. In one example, the display device 3012 may include a touch screen allowing for user-interaction with the smart device 3008 via the display device. The smart device 3008 may be located anywhere within or on the patch 3000. The smart device 3008 of FIG. 30 may be included in any example of a wrap or patch as described herein in connection with FIGS. 1 through 40.

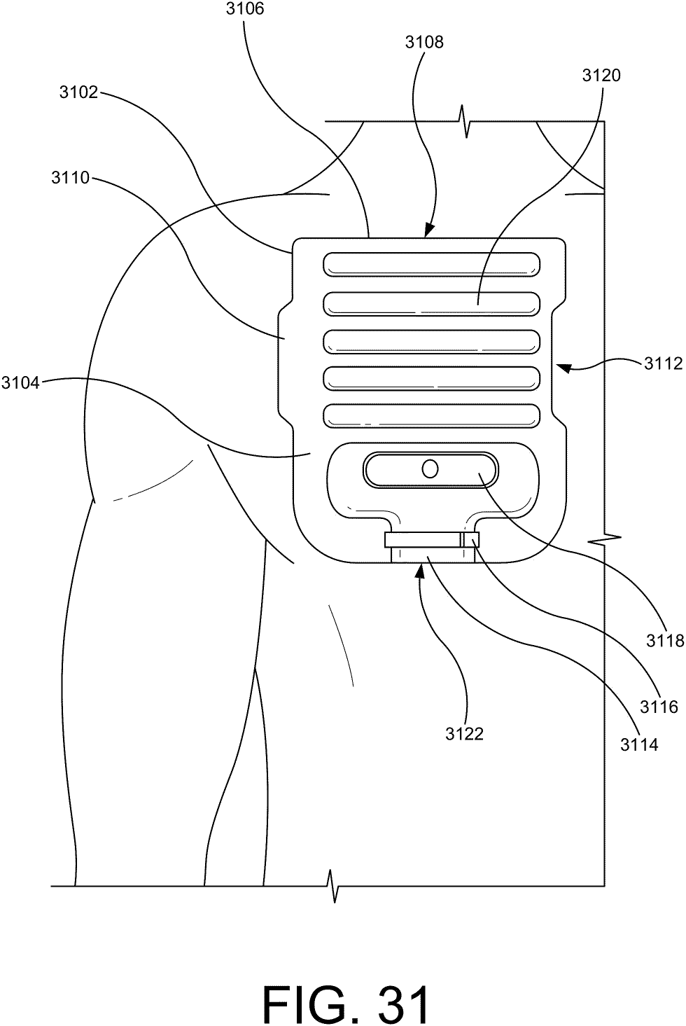

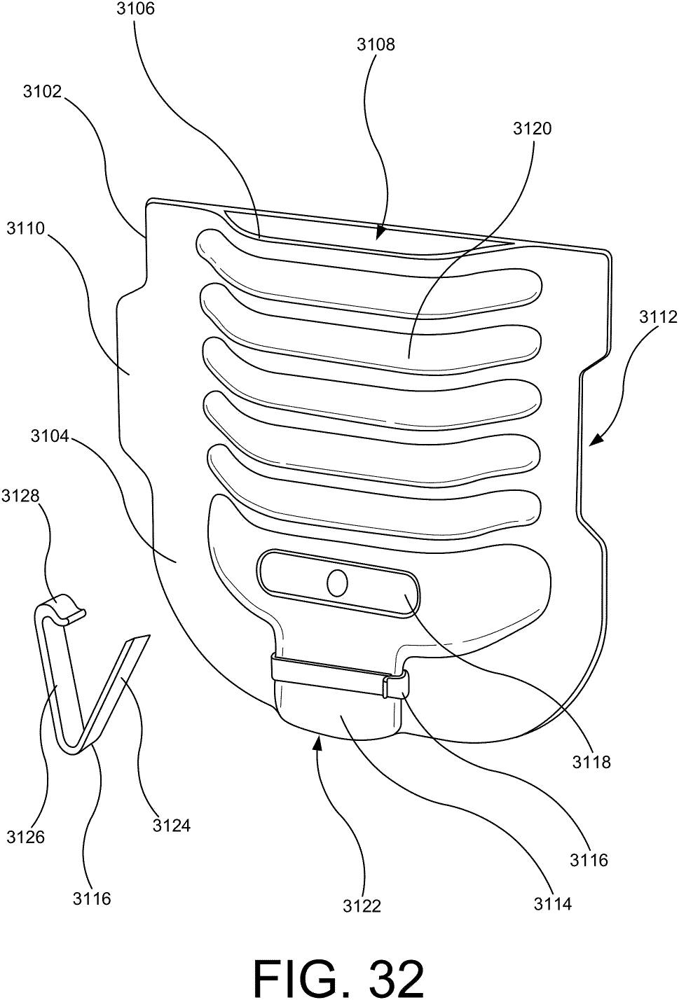

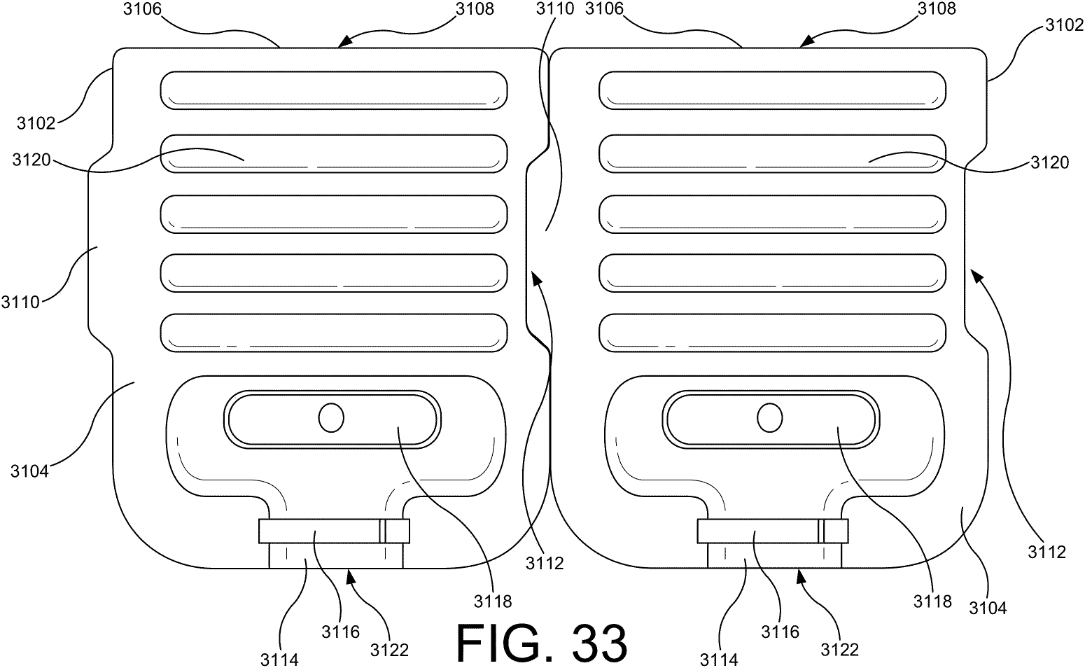

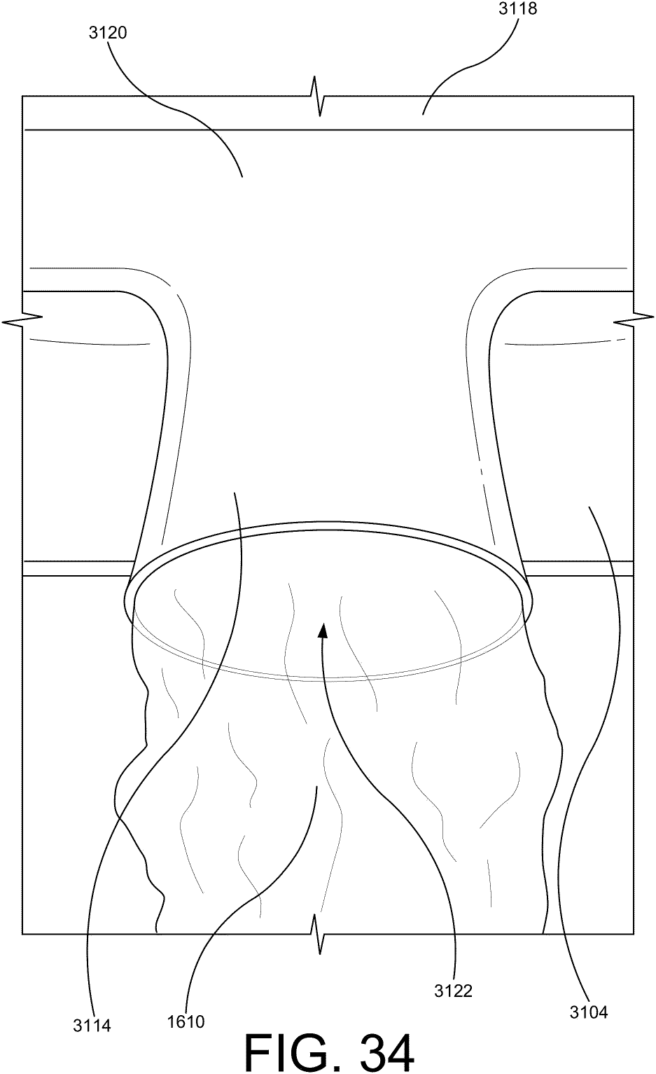

FIG. 31 illustrates a front view of a patch 3102, according to an example of the principles described herein. FIG. 32 illustrates a front perspective view of the patch 3102 of FIG. 31 including a retention clip 3116, according to an example of the principles described herein. FIG. 33 illustrates a front perspective view of a pair of patches of FIG. 31 coupled together, according to an example of the principles described herein. FIG. 34 illustrates a close-up, front perspective view of the patch of FIG. 31 with a retention clip 3116 removed, according to an example of the principles described herein. The patch 3102 of FIGS. 31 through 34 may include a reservoir 3120 coupled to or monolithically formed with a flange 3104. A first via 3108 defined in an inlet 3106 may be fluidically coupled to the reservoir 3120 to allow for a therapeutic fluid 1610 to be poured into the reservoir 3120.

An adhesive may be placed on a side of the flange 3104 to at least temporarily couple the patch 3102 to the tissues of the animal. A tab 3110 may be formed along a portion of the flange 3104. The tab 3110 may not have adhesive to allow the tab 3110 to be used to overcome the adhesive and separate the patch 3102 from the tissues of the animal. On a second side of the patch 3102, an indention 3112 may be defined in the flange 3104 to allow for two patches 3102 to nest juxtaposition to one another as depicted in FIG. 33. In this manner, one or more patches 3102 may be applied to the issues of the animal along a larger area.

The patch 3102 may further include second via 3122 defined in an outlet 3114 that fluidically couples the reservoir 3120 to the outlet 3114 and which is located opposite the inlet 3106. The second via 3122 of the outlet 3114 allows for the therapeutic fluid 1610 to be evacuated from the reservoir 3120. In one example, the therapeutic fluid 1610 may be restricted from exiting the patch 3102 via the outlet 3114 through application of a closure device such as a clip 3116. In one example, the clip 3116 may extend around an extended portion of the outlet 3114. In one example, the clip 3116 may include a piece of plastic formed with a natural spring such that the clip 3116 has an open resting state as depicted in FIG. 32 such that when the clip 3116 is engaged, a first arm the clip 3116 applies a force against a hook 3128 formed on a second arm 3126 of the clip 3116 and locks the first arm 3124 in an engaged state with the hook 3128. As depicted in FIG. 34, once the clip 3116 is removed from the outlet 3114, the therapeutic fluid 1610 may be evacuated from the reservoir 3120.

In one example, the patch 3102 may include a smart device 3118 coupled to or formed with the reservoir 3120 and may include the elements described above in connection with the smart device 3008 of the example of FIG. 30. In one example, the smart device 3118 may include a vibration motor in a hard casing to provide additional stimulation to the tissues of the animals as the tissues are being treated through application of the therapeutic fluid 1610. In one example, the smart device 3118 may be embedded in the patch 3102 and removed from the patch 3102 by deforming a portion of the patch 3102 and “popping out” the smart device 3118. Reengagement of the smart device 3118 may be achieved by applying pressure to the smart device 3118 as it sits atop the area in which it is to be seated and “popping in” the smart device 3118 into a seated position. In one example, the area in which the smart device 3118 is to be seated may include one or more lips, cusps, ledges, clips, or fasteners to contain the smart device 3118. In one example, the smart device 3118 may include a power source such as one or more batteries. Thus, the removal of the smart device 3118 from the patch 3102 may allow for the recharging or replacing of the power source.

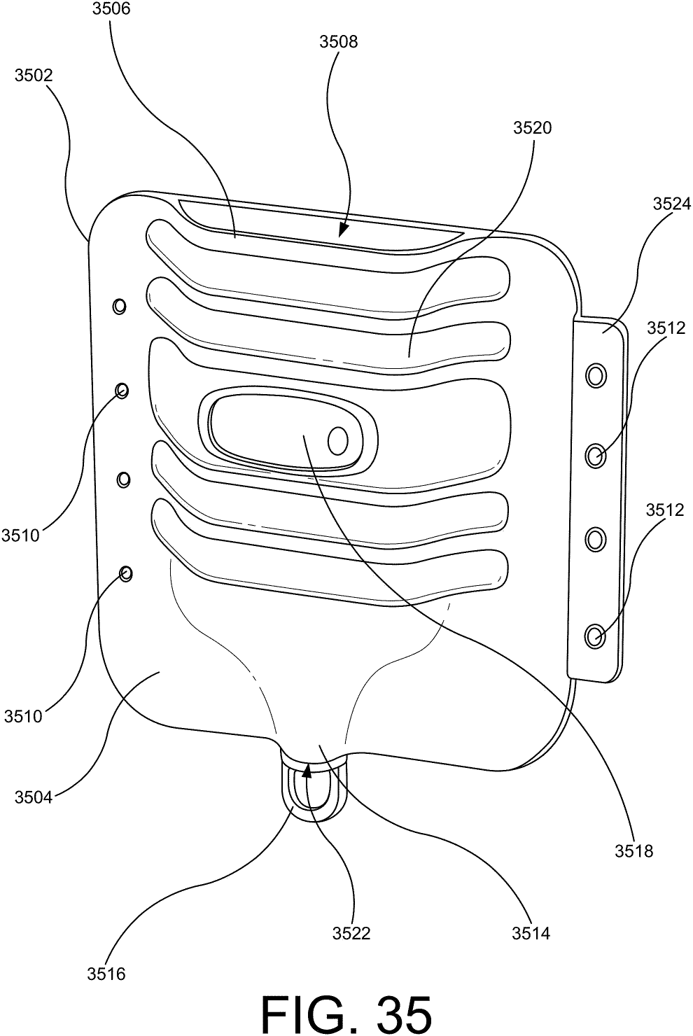

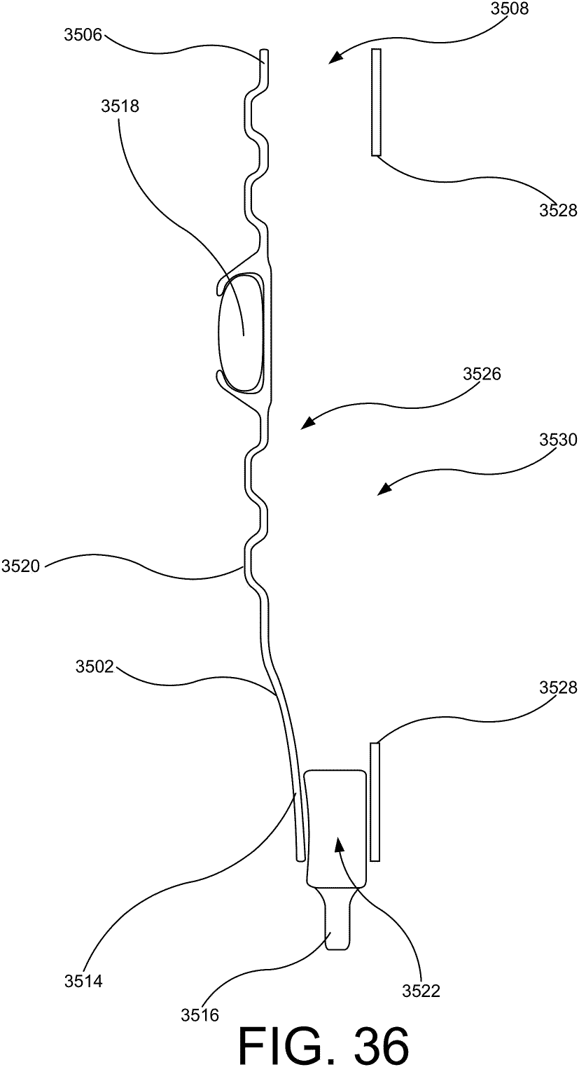

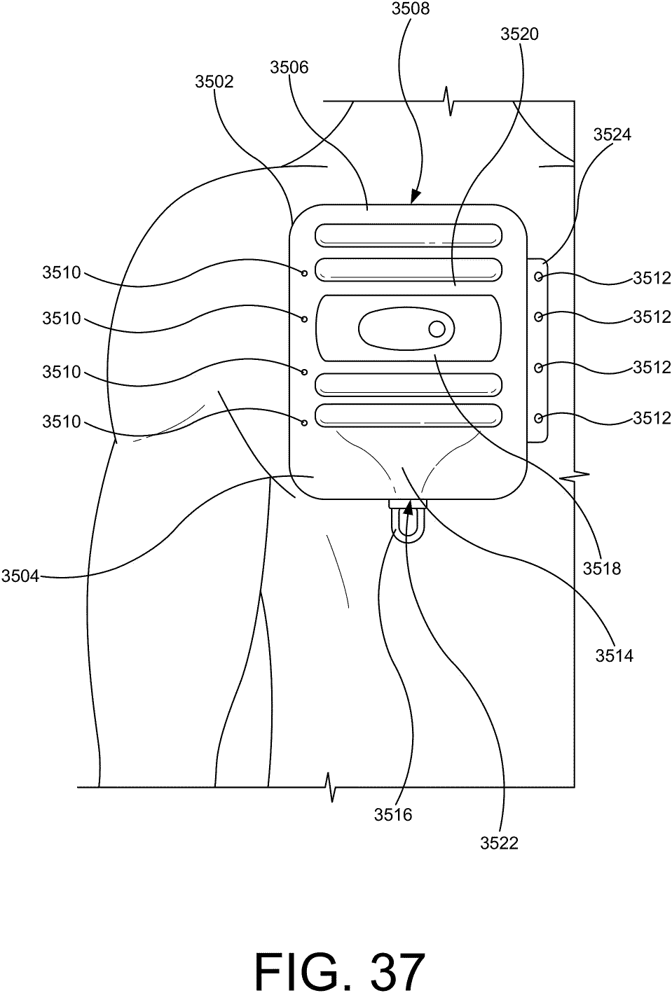

FIG. 35 illustrates a front perspective view of a patch 3502, according to an example of the principles described herein. FIG. 36 illustrates a side, cutaway view of the patch 3502 of FIG. 35, according to an example of the principles described herein. FIG. 37 illustrates front view of the patch 3502 of FIG. 35, according to an example of the principles described herein. The patch 3502 of FIGS. 35 through 37 may include a reservoir 3520 coupled to or monolithically formed with a flange 3504. A first via 3508 defined in an inlet 3506 may be fluidically coupled to the reservoir 3520 to allow for a therapeutic fluid 1610 to be poured into the reservoir 3520. The reservoir 3520 may define a cavity 3526 in which the therapeutic fluid 1610 may be retained. A back portion of the patch 3502 may include an edge 3528 that defines an opening 3530. The opening 3530 allows for the therapeutic fluid 1610 to directly contact the tissues of the animal when the patch 3502 is coupled to the tissues and the therapeutic fluid 1610 is introduced into the cavity 3526 of the reservoir 3520. An adhesive may be placed on a side of the flange 3504 to at least temporarily couple the patch 3502 to the tissues of the animal.

In one example, one or more the patches 3502 may be coupled to one another via one or more pins 3512 affixed to a tab 3524 located on a first side of a first patch 3502 engaging with one or more apertures 3510 defined in a second side of a second patch 3502. In this manner, the patches 3502 may be coupled together and juxtaposition to one another as similarly depicted in FIG. 33. In one example, the tab 3524 may be coupled to or monolithically formed with the flange 3504 but extending on a separate plane relative to the flange 3504 such that the tab 3524 does not sit flush with the tissues of the animal as the flange 3504 but extends above the tissues. This may allow for the pins 3512 to extend below the tab 3524 and engage with the apertures 3510 from the front of the patch 3502.

The patch 3502 may further include second via 3522 defined in an outlet 3514 that fluidically couples the reservoir 3520 to the outlet 3514 and which is located opposite the inlet 3506. The second via 3522 of the outlet 3514 allows for the therapeutic fluid 1610 to be evacuated from the reservoir 3520. In one example, the therapeutic fluid 1610 may be restricted from exiting the patch 3502 via the outlet 3514 through application of a closure device such as a stop 3516. In one example, the stop 3516 may couple to the second via 3522 of the outlet 3514 through an interference fit, through a mating threaded engagement, or other coupling method. Once the stop 3516 is removed from the outlet 3514, the therapeutic fluid 1610 may be evacuated from the cavity 3526 of the reservoir 3520.

In one example, the patch 3502 may include a smart device 3518 coupled to or formed with the reservoir 3520 and may include the elements described above in connection with the smart device 3008 of the example of FIG. 30. In one example, the smart device 3518 may include a vibration motor in a hard casing to provide additional stimulation to the tissues of the animals as the tissues are being treated through application of the therapeutic fluid 1610. In one example, the smart device 3518 may be embedded in the patch 3502 and removed from the patch 3502 by deforming a portion of the patch 3502 and “popping out” the smart device 3518. Reengagement of the smart device 3518 may be achieved by applying pressure to the smart device 3518 as it sits atop the area in which it is to be seated and “popping in” the smart device 3518 into a seated position.

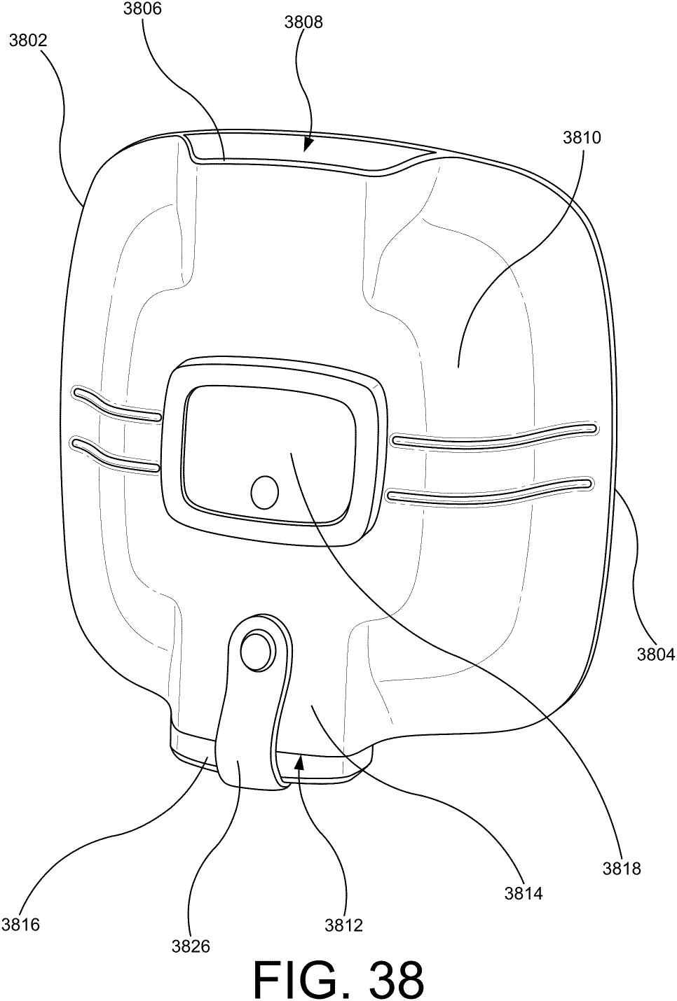

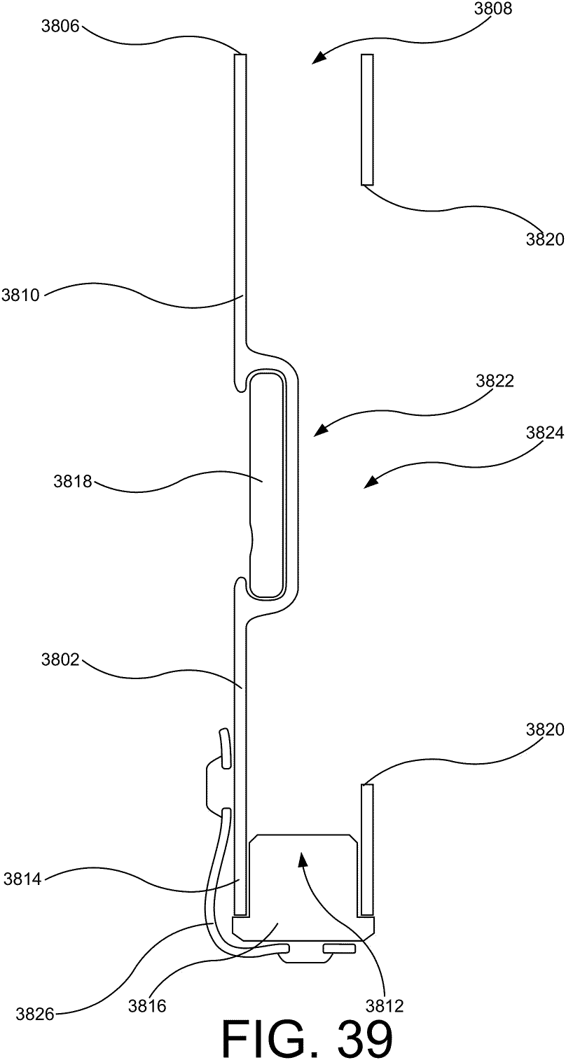

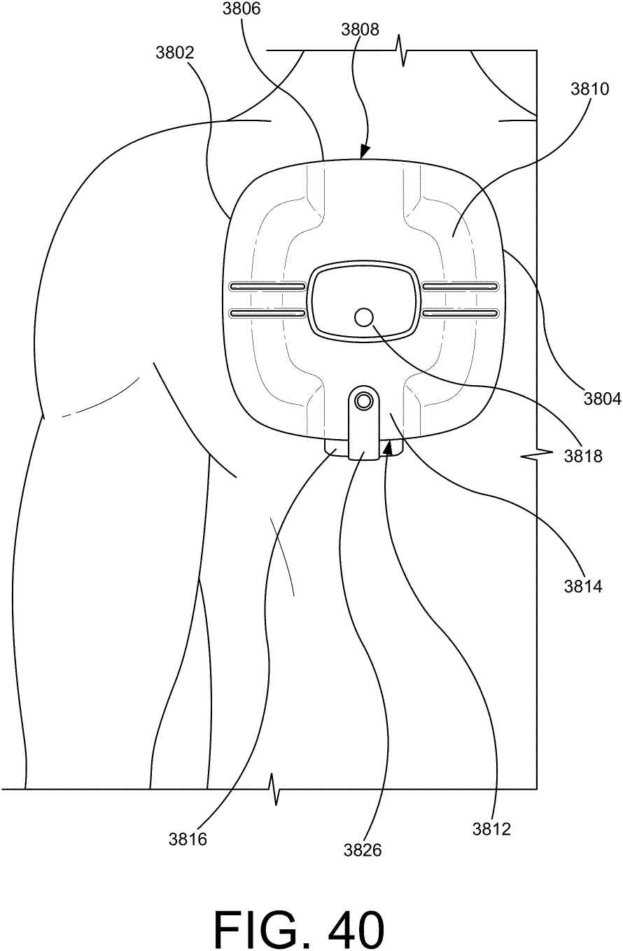

FIG. 38 illustrates a front perspective view of a patch 3802, according to an example of the principles described herein. FIG. 39 illustrates a side, cutaway view of the patch 3802 of FIG. 38, according to an example of the principles described herein. FIG. 40 illustrates front view of the patch 3802 of FIG. 38, according to an example of the principles described herein. The patch 3802 of FIGS. 38 through 40 may include a reservoir 3810. A first via 3808 defined in an inlet 3806 may be fluidically coupled to the reservoir 3810 to allow for a therapeutic fluid 1610 to be poured into the reservoir 3810. The reservoir 3810 may define a cavity 3822 in which the therapeutic fluid 1610 may be retained. A back portion of the patch 3802 may include an edge 3820 that defines an opening 3824. The opening 3824 allows for the therapeutic fluid 1610 to directly contact the tissues of the animal when the patch 3802 is coupled to the tissues and the therapeutic fluid 1610 is introduced into the cavity 3822 of the reservoir 3810. An adhesive may be placed on a side of a base 3804 to at least temporarily couple the patch 3802 to the tissues of the animal.

The patch 3802 may further include second via 3812 defined in an outlet 3814 that fluidically couples the reservoir 3810 to the outlet 3814 and which is located opposite the inlet 3806. The second via 3812 of the outlet 3814 allows for the therapeutic fluid 1610 to be evacuated from the reservoir 3810. In one example, the therapeutic fluid 1610 may be restricted from exiting the patch 3802 via the outlet 3814 through application of a closure device such as a stop 3816. In one example, the stop 3816 may couple to the second via 3812 of the outlet 3814 through an interference fit or other coupling method. Once the stop 3816 is removed from the outlet 3814, the therapeutic fluid 1610 may be evacuated from the cavity 3822 of the reservoir 3810. In one example, the stop 3816 may be coupled to the patch 3802 via a tether 3826 so that the stop 3816 may not be lost as it is removed from the outlet 3814.

In one example, the patch 3802 may include a smart device 3818 coupled to or formed with the reservoir 3810 and may include the elements described above in connection with the smart device 3008 of the example of FIG. 30. In one example, the smart device 3818 may include a vibration motor in a hard casing to provide additional stimulation to the tissues of the animals as the tissues are being treated through application of the therapeutic fluid 1610. In one example, the smart device 3818 may be embedded in the patch 3802 and removed from the patch 3802 by deforming a portion of the patch 3802 and “popping out” the smart device 3818. Reengagement of the smart device 3818 may be achieved by applying pressure to the smart device 3818 as it sits atop the area in which it is to be seated and “popping in” the smart device 3818 into a seated position.

Although the examples of FIGS. 1 through 40 are depicted as being used in connection with a human subject and a horse, the examples described herein may be utilized to provide therapeutic treatment to any animal. Further, the various elements of the examples of FIGS. 1 through 40 may be includes in any given example in any combination.

### Conclusion

The examples described herein provide systems, devices, and methods of treating tissues of an animal using a nan-sized ice particle slurry within a wrap or patch. The wraps and patches described herein prevent any further tissue damage to the treated tissues while still enabling an ice to skin contact.

While the present systems and methods are described with respect to the specific examples, it is to be understood that the scope of the present systems and methods are not limited to these specific examples. Since other modifications and changes varied to fit particular operating requirements and environments will be apparent to those skilled in the art, the present systems and methods are not considered limited to the example chosen for purposes of disclosure, and covers all changes and modifications which do not constitute departures from the true spirit and scope of the present systems and methods.

Although the application describes examples having specific structural features and/or methodological acts, it is to be understood that the claims are not necessarily limited to the specific features or acts described. Rather, the specific features and acts are merely illustrative of some examples that fall within the scope of the claims of the application.

What is claimed is:

1. A therapeutic device comprising:
a substrate;
a seal to couple the substrate to a surface; and
at least one port defined in the substrate through which a therapeutic fluid is introduced between the surface and the substrate such that the therapeutic fluid directly contacts the surface.

2. The therapeutic device of claim 1, wherein the surface is outer tissue of a vertebrate animal.

3. The therapeutic device of claim 1, wherein the therapeutic fluid comprises a nano-dimensioned ice crystal slurry.

4. The therapeutic device of claim 1, wherein the seal comprises a suction device that creates negative fluid pressure between the substrate and the surface to create a partial vacuum between the substrate and the surface.

5. The therapeutic device of claim 4, further comprising a pressure regulator to selectively release pressure within the suction device.

6. The therapeutic device of claim 1, wherein the substrate is made of an elastic material to conform to a contour shape of the surface.

7. The therapeutic device of claim 1, wherein the at least one port comprises:
a first port through which the therapeutic fluid is introduced between the surface and the substrate; and
a second port through which the therapeutic fluid is drainable from between the surface and the substrate.

8. The therapeutic device of claim 1, further comprising a temperature readout device disposed on the substrate.

9. The therapeutic device of claim 1, further comprising a notification device to produce one or more notifications associated with use of the therapeutic device.

10. The therapeutic device of claim 1, further comprising:
at least one sensor disposed on the substrate; and
a communication device to communicate data obtained from the sensor.

11. The therapeutic device of claim 1, further comprising at least one coupling device to couple the therapeutic device to another therapeutic device.

12. A therapeutic method, comprising:
applying a therapeutic device to a surface, the therapeutic device comprising:
a substrate;
a seal to couple the substrate to the surface; and
at least one port defined in the substrate through which a therapeutic fluid is introduced between the surface and the substrate such that the therapeutic fluid directly contacts the surface; and
introducing the therapeutic fluid between the surface and the substrate via the at least one port.

13. The therapeutic method of claim 12, further comprising coupling the substrate to the surface via the seal, the seal comprising, a suction device, an adhesive, hook and loop, tape, and combinations thereof.

14. The therapeutic method of claim 13, further comprising creating negative fluid pressure between the substrate and the surface to create a partial vacuum between the substrate and the surface.

15. The therapeutic method of claim 14, further comprising, with a pressure regulator, selectively releasing pressure within the suction device.

16. The therapeutic method of claim 12, further comprising forming the therapeutic fluid, the therapeutic fluid comprising a nano-dimensioned ice crystal slurry.

17. The therapeutic method of claim 12, wherein the at least one port comprises:
a first port through which the therapeutic fluid is introduced between the surface to be treated and the substrate; and
a second port through which the therapeutic fluid is drained from between the surface to be treated and the substrate,
the therapeutic method further comprising removing the therapeutic fluid via the second port.

18. The therapeutic method of claim 12, further comprising:
detecting, via at least one sensor coupled to the therapeutic device, at least one environmental characteristic associated with the therapeutic method; and
transmitting, via a communication device associated with the therapeutic device, data defining the at least one environmental characteristic.

19. The therapeutic method of claim 18, wherein the at least one sensor comprises a temperature sensor, the communication device transmitting data defining a temperature of the therapeutic fluid.

20. The therapeutic method of claim 12, further comprising coupling the therapeutic device to another therapeutic device via at least one coupling device.
